SEQUENCE LISTING (90 sequences)

SEQ ID NO: 1 - DNA, 768 bp, Staphylococcus simulans
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcaccaccaccaccaccactga

SEQ ID NO: 2 - protein, 255 aa, Staphylococcus simulans
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEHHHHHH

SEQ ID NO: 3 - DNA, 1512 bp, Phage K
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagcaccaccaccaccaccactga

SEQ ID NO: 4 - protein, 503 aa, Phage K
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLEHHHHHH

SEQ ID NO: 5 - DNA, 1941 bp, Artificial Sequence
  Phage K & Staphylococcus simulans
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcaggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcaagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcaccaccaccaccaccactga

SEQ ID NO: 6 - protein, 646 aa, Artificial Sequence
  Phage K & Staphylococcus simulans
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEHHHHHH

SEQ ID NO: 7 - DNA, 1974 bp, Artificial Sequence
  Staphylococcus simulans & Phage K
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtctcgacgctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtctcgacacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcaccaccaccaccaccactga

SEQ ID NO: 8 - protein, 657 aa, Artificial Sequence
  Staphylococcus simulans & Phage K
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGLDAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEHHHHHH

SEQ ID NO: 9 - DNA, 822 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcgccagattaaaatttggtttcagaaccgccgcatgaaatggaaaaaagtcgagcaccaccaccaccaccactga

SEQ ID NO: 10 - protein, 273 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLERQIKIWFQNRRMKWKKVEHHHHHH

SEQ ID NO: 11 - DNA, 864 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagtgggaagcgaaactggcgaaagcgctggcgaaagcgctggcgaaacatctggcgaaagcgctggcgaaagcgctgaaagcgtgcgaagcggtcgagcaccaccaccaccaccactga

SEQ ID NO: 12 - protein, 287 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEWEAKLAKALAKALAKHLAKALAKALKACEAVEHHHHHH

SEQ ID NO: 13 - DNA, 840 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagatggtgaccgtgctgtttcgccgcctgcgcattcgccgcgcgagcggcccgccgcgcgtgcgcgtggtcgagcaccaccaccaccaccactga

SEQ ID NO: 14 - protein, 279 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEMVTVLFRRLRIRRASGPPRVRVVEHHHHHH

SEQ ID NO: 15 - DNA, 828 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagaaactggcgctgaaactggcgctgaaagcgctgaaagcggcgctgaaactggcggtcgagcaccaccaccaccaccactga

SEQ ID NO: 16 - protein, 275 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEKLALKLALKALKAALKLAVEHHHHHH

SEQ ID NO: 17 - DNA, 810 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcgccgccagcgccgcaccagcaaactgatgaaacgcgtcgagcaccaccaccaccaccactga

SEQ ID NO: 18 - protein, 269 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLERRQRRTSKLMKRVEHHHHHH

SEQ ID NO: 19 - DNA, 828 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagctgctgattattctgcgccgccgcattcgcaaacaggcgcatgcgcatagcaaagtcgagcaccaccaccaccaccactga

SEQ ID NO: 20 - protein, 275 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLELLIILRRRIRKQAHAHSKVEHHHHHH

SEQ ID NO: 21 - DNA, 798 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcgccgccgccgccgccgccgccgcgtcgagcaccaccaccaccaccactga

SEQ ID NO: 22 - protein, 265 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLERRRRRRRRVEHHHHHH

SEQ ID NO: 23 - DNA, 813 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagggccgcaaaaaacgccgccagcgccgccgcccgccgcaggtcgagcaccaccaccaccaccactga

SEQ ID NO: 24 - protein, 270 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEGRKKRRQRRRPPQVEHHHHHH

SEQ ID NO: 25 - DNA, 855 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagggctggaccctgaacagcgcgggctatctgctgggcaaaattaacctgaaagcgctggcggcgctggcgaaaaaaattctggtcgagcaccaccaccaccaccactga

SEQ ID NO: 26 - protein, 284 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEGWTLNSAGYLLGKINLKALAALAKKILVEHHHHHH

SEQ ID NO: 27 - DNA, 837 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgaggcgggctatctgctgggcaaaattaacctgaaagcgctggcggcgctggcgaaaaaaattctggtcgagcaccaccaccaccaccactga

SEQ ID NO: 28 - protein, 278 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEAGYLLGKINLKALAALAKKILVEHHHHHH

SEQ ID NO: 29 - DNA, 795 bp, Artificial Sequence
  Fusion Protein- Staphylococcus simulans- Synthetic
atggctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagcgcaaaaaacgccgccagcgccgccgcctcgagcaccaccaccaccaccactga

SEQ ID NO: 30 - protein, 264 aa, Artificial Sequence
  Fusion Protein- Staphylococcus simulans- Synthetic
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKRKKRRQRRRLEHHHHHH

SEQ ID NO: 31 - DNA, 1566 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagcgccagattaaaatttggtttcagaaccgccgcatgaaatggaaaaaagtcgagcaccaccaccaccaccactga

SEQ ID NO: 32 - protein, 521 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLERQIKIWFQNRRMKWKKVEHHHHHH

SEQ ID NO: 33 - DNA, 1608 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagtgggaagcgaaactggcgaaagcgctggcgaaagcgctggcgaaacatctggcgaaagcgctggcgaaagcgctgaaagcgtgcgaagcggtcgagcaccaccaccaccaccactga

SEQ ID NO: 34 - protein, 535 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLEWEAKLAKALAKALAKHLAKALAKALKACEAVEHHHHHH

SEQ ID NO: 35 - DNA, 1584 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagatggtgaccgtgctgtttcgccgcctgcgcattcgccgcgcgagcggcccgccgcgcgtgcgcgtggtcgagcaccaccaccaccaccactga

SEQ ID NO: 36 - protein, 527 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLEMVTVLFRRLRIRRASGPPRVRVVEHHHHHH

SEQ ID NO: 37 - DNA, 1572 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagaaactggcgctgaaactggcgctgaaagcgctgaaagcggcgctgaaactggcggtcgagcaccaccaccaccaccactga

SEQ ID NO: 38 - protein, 523 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLEKLALKLALKALKAALKLAVEHHHHHH

SEQ ID NO: 39 - DNA, 1566 bp, Artificial Sequence; identical to SEQ ID NO: 31
  Fusion Protein- Phage K- Synthetic

SEQ ID NO: 40 - protein, 521 aa, Artificial Sequence; identical to SEQ ID NO: 32
  Fusion Protein- Phage K- Synthetic

SEQ ID NO: 41 - DNA, 1554 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagcgccgccagcgccgcaccagcaaactgatgaaacgcgtcgagcaccaccaccaccaccactga

SEQ ID NO: 42 - protein, 517 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLERRQRRTSKLMKRVEHHHHHH

SEQ ID NO: 43 - DNA, 1572 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagctgctgattattctgcgccgccgcattcgcaaacaggcgcatgcgcatagcaaagtcgagcaccaccaccaccaccactga

SEQ ID NO: 44 - protein, 523 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLELLIILRRRIRKQAHAHSKVEHHHHHH

SEQ ID NO: 45 - DNA, 1542 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagcgccgccgccgccgccgccgccgcgtcgagcaccaccaccaccaccactga

SEQ ID NO: 46 - protein, 513 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLERRRRRRRRVEHHHHHH

SEQ ID NO: 47 - DNA, 1557 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagggccgcaaaaaacgccgccagcgccgccgcccgccgcaggtcgagcaccaccaccaccaccactga

SEQ ID NO: 48 - protein, 518 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLEGRKKRRQRRRPPQVEHHHHHH

SEQ ID NO: 49 - DNA, 1599 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgagggctggaccctgaacagcgcgggctatctgctgggcaaaattaacctgaaagcgctggcggcgctggcgaaaaaaattctggtcgagcaccaccaccaccaccactga

SEQ ID NO: 50 - protein, 532 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLEGWTLNSAGYLLGKINLKALAALAKKILVEHHHHHH

SEQ ID NO: 51 - DNA, 1581 bp, Artificial Sequence
  Fusion Protein- Phage K- Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaactcgaggcgggctatctgctgggcaaaattaacctgaaagcgctggcggcgctggcgaaaaaaattctggtcgagcaccaccaccaccaccactga

SEQ ID NO: 52 - protein, 526 aa, Artificial Sequence
  Fusion Protein- Phage K- Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKLEAGYLLGKINLKALAALAKKILVEHHHHHH

SEQ ID NO: 53 - DNA, 1995 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcgccagattaaaatttggtttcagaaccgccgcatgaaatggaaaaaagtcgagcaccaccaccaccaccactga

SEQ ID NO: 54 - protein, 664 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLERQIKIWFQNRRMKWKKVEHHHHHH

SEQ ID NO: 55 - DNA, 2037 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagtgggaagcgaaactggcgaaagcgctggcgaaagcgctggcgaaacatctggcgaaagcgctggcgaaagcgctgaaagcgtgcgaagcggtcgagcaccaccaccaccaccactga

SEQ ID NO: 56 - protein, 678 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEWEAKLAKALAKALAKHLAKALAKALKACEAVEHHHHHH

SEQ ID NO: 57 - DNA, 2013 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagatggtgaccgtgctgtttcgccgcctgcgcattcgccgcgcgagcggcccgccgcgcgtgcgcgtggtcgagcaccaccaccaccaccactga

SEQ ID NO: 58 - protein, 670 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEMVTVLFRRLRIRRASGPPRVRVVEHHHHHH

SEQ ID NO: 59 - DNA, 2001 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagaaactggcgctgaaactggcgctgaaagcgctgaaagcggcgctgaaactggcggtcgagcaccaccaccaccaccactga

SEQ ID NO: 60 - protein, 666 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEKLALKLALKALKAALKLAVEHHHHHH

SEQ ID NO: 61 - DNA, 1983 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcaggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcaagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcgccgccagcgccgcaccagcaaactgatgaaacgcgtcgagcaccaccaccaccaccactga

SEQ ID NO: 62 - protein, 660 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLERRQRRTSKLMKRVEHHHHHH

SEQ ID NO: 63 - DNA, 2001 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcaggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcaagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagctgctgattattctgcgccgccgcattcgcaaacaggcgcatgcgcatagcaaagtcgagcaccaccaccaccaccactga

SEQ ID NO: 64 - protein, 666 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLELLIILRRRIRKQAHAHSKVEHHHHHH

SEQ ID NO: 65 - DNA, 1971 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcaggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcaagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagcgccgccgccgccgccgccgccgcgtcgagcaccaccaccaccaccactga

SEQ ID NO: 66 - protein, 656 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLERRRRRRRRVEHHHHHH

SEQ ID NO: 67 - DNA, 1986 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcaggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcaagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagggccgcaaaaaacgccgccagcgccgccgcccgccgcaggtcgagcaccaccaccaccaccactga

SEQ ID NO: 68 - protein, 661 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEGRKKRRQRRRPPQVEHHHHHH

SEQ ID NO: 69 - DNA, 2028 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgagggctggaccctgaacagcgcgggctatctgctgggcaaaattaacctgaaagcgctggcggcgctggcgaaaaaaattctggtcgagcaccaccaccaccaccactga

SEQ ID NO: 70 - protein, 675 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEGWTLNSAGYLLGKINLKALAALAKKILVEHHHHHH

SEQ ID NO: 71 - DNA, 2010 bp, Artificial Sequence
  Fusion Protein Phage K -Staph simulans -Synthetic
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtgatggaacaggagcaaactcgggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcgagttctacagtagttaaagatggtaaaacaagtagcgcaagtctcgacgctgcaacacatgaacattcagcacaatggttgaataattacaaaaaaggatatggttacggcccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataagtctactaatactctgggtgttctgtggggaactataaagctcgaggcgggctatctgctgggcaaaattaacctgaaagcgctggcggcgctggcgaaaaaaattctggtcgagcaccaccaccaccaccactga

SEQ ID NO: 72 - protein, 669 aa, Artificial Sequence
  Fusion Protein Phage K -Staph simulans -Synthetic
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASLDAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKLEAGYLLGKINLKALAALAKKILVEHHHHHH

SEQ ID NO: 73 - DNA, 39 bp, Artificial Sequence
  Chemically Synthesized
acgtacgtcatatggctgcaacacatgaacattcagcac

SEQ ID NO: 74 - DNA, 32 bp, Artificial Sequence
  Chemically Synthesized
gcgctactcgagaccacctgcttttccatatc

SEQ ID NO: 75 - DNA, 37 bp, Artificial Sequence
  Chemically Synthesized
atcatcgtcgacgctgcaacacatgaacattcagcac

SEQ ID NO: 76 - DNA, 24 bp, Artificial Sequence
  Chemically Synthesized
gtttgtctcgagacctgtattcgg

SEQ ID NO: 77 - DNA, 30 bp, Artificial Sequence
  Chemically Synthesized
gcgcatctcgagacagtaactccaacgccg

SEQ ID NO: 78 - DNA, 25 bp, Artificial Sequence
  Chemically Synthesized
cgtttagaggccccaaggggttatg

SEQ ID NO: 79 - DNA, 22 bp, Artificial Sequence
  Chemically Synthesized
gatatagtcgacgctaagactc

SEQ ID NO: 80 - protein, 16 aa, Artificial Sequence
  Chemically Synthesized
RQIKIWFQNRRMKWKK

SEQ ID NO: 81 - protein, 31 aa, Artificial Sequence
  Chemically Synthesized
WEEAKLAKALAKALAKHLAKALAKALKACEA

SEQ ID NO: 82 - protein, 22 aa, Artificial Sequence
  Chemically Synthesized
MVTVLFRRLRIRRASGPPRVRV

SEQ ID NO: 83 - protein, 18 aa, Artificial Sequence
  Chemically Synthesized
KLALKLALKALKAALKLA

SEQ ID NO: 84 - protein, 12 aa, Artificial Sequence
  Chemically Synthesized
RRQRRTSKLMKR

SEQ ID NO: 85 - protein, 18 aa, Artificial Sequence
  Chemically Synthesized
LLIILRRRIRKQAHAHSK

SEQ ID NO: 86 - protein, 8 aa, Artificial Sequence
  Chemically Synthesized
RRRRRRRR

SEQ ID NO: 87 - protein, 13 aa, Artificial Sequence
  Chemically Synthesized
GRKKRRQRRRPPQ

SEQ ID NO: 88 - protein, 27 aa, Artificial Sequence
  Chemically Synthesized
GWTLNSAGYLLGKINLKALAALAKKIL

SEQ ID NO: 89 - protein, 21 aa, Artificial Sequence
  Chemically Synthesized
AGYLLGKINLKALAALAKKIL

SEQ ID NO: 90 - protein, 9 aa, Artificial Sequence
  Chemically Synthesized
RKKRRQRRR

(19) United States
(12) Patent Application Publication
(10) Pub. No.: US 2016/0097044 A1
(43) Pub. Date: Apr. 7, 2016

(54) Antimicrobial Enzyme Fusions Reduce Resistance and Kill Intracellular Staphylococcus aureus

(71) Applicant: The United States of America, as Represented by the Secretary of Agriculture, Washington, DC (US)

(72) Inventor: David M. Donovan, Baltimore, MD (US)

(21) Appl. No.: 14/503,397

(22) Filed: Oct. 1, 2014

(51) Int. Cl. C12N 9/52; A01N 63/00; C07K 7/08; C12N 9/80; C12N 9/50

(52) U.S. Cl. CPC C12N 9/52; C12N 9/80; C12N 9/50; C07K 7/08; A01N 63/00; C12Y 305/01028; C12Y 304/24075; C07K 2319/33; A61K 38/00

(57) ABSTRACT

Multi-drug resistant bacteria are a persistent problem in modern health care, food safety and animal health. There is a need for new antimicrobials to replace over-used conventional antibiotics. Here we describe engineered triple-acting staphylolytic peptidoglycan hydrolases wherein three unique antimicrobial activities from two parental proteins are combined into a single fusion protein, effectively reducing the incidence of resistant strain development. The fusion protein reduced colonization by S. aureus in a rat nasal colonization model, surpassing the efficacy of either parental protein. Modification of the triple-acting lytic construct with a protein transduction domain significantly enhanced both biofilm eradication and the ability to kill intracellular Staphylococcus aureus as demonstrated in cultured cells, and mouse models of staphylococcal mastitis and osteomyelitis. Bacterial cell wall degrading enzyme antimicrobials can be engineered to enhance their value as potent therapeutics.

### BACKGROUND OF THE INVENTION

1. Field of the Invention

This invention relates to a pathogen-specific triple fusion construct comprising three antimicrobial domains each having a unique lytic activity: a LysK endopeptidase domain, a LysK amidase domain, and a lysostaphin glycyl-glycine endopeptidase domain. The constructs further comprise a cell wall binding domain and a protein transduction domain (PTD). Triple Fusion-PTD constructs are refractory to resistance development and can be used to kill antibiotic-resistant staphylococcal strains, to treat extracellular and intracellular S. aureus infections, and to eradicate biofilms.

2. Description of the Relevant Art

Staphylococcus aureus is an opportunistic bacterial pathogen responsible for a diverse spectrum of animal and human diseases including mastitis, osteomyelitis, and endocarditis (Lowy, F. D. 1998. New England J. Med. 339:520-532). S. aureus rapidly develops resistance to antibiotics, as illustrated by multi-drug resistant (MDR), methicillin-resistant S. aureus (MRSA) and the reduced susceptibility to vancomycin (vancomycin-intermediate strains) (Appelbaum, P. C. 2007. Clin. Infect. Dis. 45 Suppl. 3:S165-170). The number of hospitalizations due to MRSA infection continues to increase with 9.7 billion dollar increased health care costs in 2005 (Klein et al. 2007. Emerg. Infect. Dis. 13:1840-1846; Klein et al. 2013. Am. J. Epidemiol. 177(7):666-674).

S. aureus, including MRSA, can asymptomatically colonize the anterior nares of healthy humans and from these reservoirs opportunistically infect the host. During an infection, S. aureus has also been reported to reside intracellularly in some host cells, e.g., human nasal epithelial cells (Plouin-Gaudon et al. 2006. Rhinology 44:249-254), bovine mammary cells (Herbert et al. 2000. FEMS Microbiol. Lett. 193:57-62; Peton and Le Loir. 2014. Infect. Genet. Evol. 21:602-615), and avian osteoblasts (Reilly et al. 2000. Bone 26:63-70). Intracellular bacteria evade most antibiotics and the host immune system, allowing them to re-emerge after treatment and infect surrounding cells and tissues (Ellington et al. 2006. J. Orthop. Res. 24:87-93). For example, S. aureus is the most common infective agent associated with reoccurring osteomyelitis (53%) in war zone blast wounds and 60% of these strains were antibiotic-resistant (Yun et al. 2008. J. Trauma 64:S163-S168). There is a need for antimicrobials that can kill both MDR and intracellular bacteria.

Peptidoglycan hydrolases (PGHs) are candidate antimicrobials with properties that are ideal for treatment of MDR infections (Donovan et al. 2009. Biotech International 21:6-10). Peptidoglycan hydrolases digest the bacterial cell wall peptidoglycan (PG) causing osmolysis and death (Becker et al. 2008. FEMS Microbiol. Lett. 287:185-191). The Gram-positive bacterial peptidoglycan structure is highly divergent (Schleifer and Kandler. 1972. Bacteriol. Rev. 36:407-477) and peptidoglycan hydrolase domains target unique bonds in the peptidoglycan and lyse target bacteria often with near-species or serovar-specificity (Schmelcher et al. 2010. Appl. Environ. Microbiol. 76:5745-5756), thus avoiding the adverse effects of antibiotic selection on unrelated commensal strains. Peptidoglycan hydrolases have been shown to be versatile in their applications (for reviews see, Schmelcher et al. 2012. Future Microbiol. 7:1147-1171; Nelson et al. 2012. Adv. Virus Res. 83:299-365; Shen et al. 2012. In: Bacteriophages in Health and Disease, eds. S. Abedon & P. Hyman, CAD Press, pp. 217-239).

The frequency of multi-drug resistant S. aureus is approaching epidemic proportions globally. There is a need to develop pathogen-specific agents that can target the bacterial cell surface, kill antibiotic-resistant strains, treat chronic intracellular and extracellular S. aureus infections and avoid resistant strain development.

### SUMMARY OF THE INVENTION

We have discovered that a triple fusion antimicrobial protein comprising three different peptidoglycan hydrolase domains each of which specifically attacks the peptidoglycan cell wall of live, untreated S. aureus from without, and further comprising a protein transduction domain, can circumvent the development of resistance and can effectively treat intracellular and extracellular S. aureus as well as planktonic and biofilm colonizations.

In accordance with this discovery, it is an object of the invention to provide a triple fusion antimicrobial protein comprising three different peptidoglycan hydrolase domains together each of which specifically targets and attacks unique bonds of the peptidoglycan cell wall of live, untreated S. aureus when exposed externally and further comprising a protein transduction domain making possible eradication of intracellular S. aureus.

It is also an object of the invention to provide a recombinant nucleic acid encoding a triple fusion antimicrobial protein comprising three different peptidoglycan hydrolase domains (1) a LysK CHAP endopeptidase, (2) a LysK amidase, and (3) a lysostaphin glycyl-glycine endopeptidase domain, a cell wall binding domain, and a protein transduction domain.

An added object of the invention is to provide a pharmaceutical composition comprising triple fusion polypeptides, a cell wall binding domain and a protein transduction domain.

An added object of the invention is to provide compositions useful for the treatment of diseases and infections caused by the extracellular and intracellular bacteria for which the LysK endolysin and lysostaphin are specific where the composition comprises a triple fusion polypeptide having three peptidoglycan hydrolase domains each of which retains its property of effectively lysing said bacteria.

An added object of the invention is method of treating diseases and infections with the triple fusion polypeptide of the invention, wherein said diseases and infections are caused by the bacteria for which the three peptidoglycan hydrolases of the triple fusion protein are highly specific.

A further object of the invention is a method of using the triple fusion polypeptide of the invention to kill S. aureus in biofilms.

Also part of this invention is a kit, comprising a composition for treatment of disease caused by the bacteria for which the LysK endolysin and lysostaphin are highly specific.

Other objects and advantages of this invention will become readily apparent from the ensuing description.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIGS. 1a-1c depict peptidoglycan hydrolase construct schematics, resistance development and effectiveness in eradicating S. aureus carriage in a rat colonization model. FIG. 1a is a schematic of the peptidoglycan hydrolase constructs. Domains: CHAP endopeptidase (CHAP, red box); N-acetylmuramoyl-L-alanine amidase (AMID, green box); M23 endopeptidase (PEP, blue oval); CBDs (LysK SH3b, gold diamond; lysostaphin SH3b, gold diamond with dot); protein transduction domain (PTD, blue circle); hexahistidine purification tag (His₆). Domains are not to scale. Specific peptidoglycan cut sites are illustrated in FIG. 3a. FIG. 1b depicts in vitro antimicrobial resistance development. Engineered triple fusions K-L and L-K suppress antimicrobial resistance development compared to LysK (K), lysostaphin (L), or a combination of equimolar concentrations of both (L+K). Changes in Minimum Inhibitory Concentrations (MIC) are depicted as a fold-change at the tenth round of sublethal exposure compared to the first exposure, with the average fold-change of 4 replicates in red. Error bars=SEM. First exposure MICs: Lysostaphin, 0.77 μg/ml (27 nM); LysK, 47 μg/ml (840 nM); Lysostaphin and LysK (L+K) in combination 0.2 μg/ml (7 nM and 3 nM, respectively); triple fusion K-L, 7 μg/ml (97 nM); triple fusion L-K, 7.8 μg/ml (107 nM). FIG. 1c depicts reduction in colonization in a rat nasal carriage model. Rats were inoculated with S. aureus strain ALR on Day 1. After 5 days, the rats were treated twice daily for 3 days with 20 μl of a 10 mg/ml solution of each enzyme. The rat noses were excised on day 10, homogenized, and quantitative cultures were performed. Each point represents the CFU recovered from an individual rat. Bars indicate the median CFU/nose recovered from treated rats. Triple fusion L-K showed a significant reduction in colonization (98%) of treated rats compared with rats treated with buffer alone. Data were compiled from five independent experiments. Lyso=commercially purchased lysostaphin (AMBI, Tarrytown, N.Y.). Statistical comparisons were made with the Mann-Whitney test. SEQ ID NOs of depicted peptidoglycan hydrolase constructs: Lysostaphin (SEQ ID NO:1); Lysostaphin-PTD1-4, 6-12 (SEQ ID NOs:9, 11, 13, 15, 17, 19, 21, 23, 25, 27, 29, respectively); LysK (SEQ ID NO:3); LysK-PTD1-11 (SEQ ID NOs: 31, 33, 35, 37, 39, 41, 43, 45, 47, 49, 51, respectively); LysK-Lysostaphin Fusion K-L (SEQ ID NO:5); K-L-PTD1-4, 6-11 (SEQ ID NOs:53, 55, 57, 59, 61, 63, 65, 67, 69, 71, respectively); Lysostaphin-LysK Fusion L-K (SEQ ID NO:7).

FIG. 2 depicts Electrospray Ionization Mass Spectrometry (ESI-MS) of purified S. aureus peptidoglycan digested with triple fusion K-L Top Panel: S. aureus peptidoglycan schematic with enzyme cut sites of the parental enzymes indicated. Bottom Panel: ESI-MS spectrum of products obtained after triple fusion K-L digestion of purified S. aureus peptidoglycan. Major peaks observed are labeled with the possible compositions and schematic structures from the model above. Lyso, mature lysostaphin; GlcNAc, N-acetylglucosamine; MurNAc, N-acetyl muramic acid; D-iGln, isoglutamine (α-amidoglutamic acid).

FIGS. 3a-3d depict peptidoglycan hydrolase purity and the S. aureus bactericidal assay. FIG. 3a shows the SDS-PAGE analysis of 5 μg His₆-tagged peptidoglycan hydrolases purified by Ni-NTA chromatography. FIG. 3b is a zymogram analysis using whole S. aureus (Newman) cells embedded within the gel matrix. FIG. 3c is a plate lysis analysis of peptidoglycan hydrolase constructs using live S. aureus (Newman) cell lawns clarified with indicated picomoles of each purified peptidoglycan hydrolase. FIG. 3d is a turbidity reduction analysis showing the linear range of enzyme concentration. A twofold dilution series of each enzyme was used in a 30 minute turbidity reduction assay on live S. aureus strain Newman cells to determine the enzyme concentrations that are within the linear range for each enzyme. Reaction conditions were 150 mM NaCl in SLB. Black, dark grey, light grey, and white bars represent peptidoglycan hydrolase constructs lysostaphin (L), LysK (K), triple fusion K-L, and triple fusion L-K, respectively, as named in FIG. 1a. Values are reported as the maximum rate ΔOD₆₀₀ₙₘ/min±SEM.

FIG. 4 shows the effect of enzyme concentration on static biofilm clearance. S. aureus SA113 biofilms were treated for 1 h with 100 μl of each enzyme individually or as equimolar mixtures of LysK and lysostaphin (white bars) in 300 mM NaCl, 50 mM NaH₂PO₄, 30% glycerol. Constructs are named as in FIG. 1a. Results are reported as the percentage reduction in biofilm compared to buffer treated biofilms. Data presented represents 4 replicates. Values represent means±standard deviation (SEM). Asterisks indicate significant differences (single factor ANOVA with t-test post hoc analysis \check{S}idák correction) as compared to LysK (K) at that concentration. Adjusted alpha p<0.026. Peptidoglycan hydrolase constructs: lysostaphin (L), LysK (K), triple fusion K-L, and triple fusion (L-K), as named in FIG. 1a.

FIGS. 5a-5e depict peptidoglycan hydrolase-protein transduction domain (PGH-PTD) eradication of intracellular S. aureus in cultured cells. Cultured cells were infected, treated with gentamicin to kill extracellular S. aureus, and then treated with the peptidoglycan hydrolases indicated. All results are standardized to the gentamicin (GENT) only control. Nomenclature of the peptidoglycan hydrolase constructs is as in FIG. 1. PTDs are listed in Table 3. Asterisks indicate statistical significance detected with single factor ANOVA (α=0.05) with paired t-test posthoc analyses α=0.05 adjusted with \check{S}idák correction for multiple comparisons (for all cases p<0.01). FIG. 5a shows the bovine mammary epithelial cell line (MAC-T) infected with strain Newbould 305 (N=8). Error bars represent SEM. 1-Sigma is commercial lysostaphin (Sigma). FIG. 5b shows human brain microvasculature epithelial cells (hBMEC) infected with S. aureus strain ISP479C (N=3). FIG. 5c shows murine primary osteoblasts (mOB) infected with S. aureus strain UAMS-1 (N=3). FIG. 5d shows a single plane of confocal microscopy z-stack overlaid on bright field exposure of live cultured MAC-T cells exposed to both S. aureus and PGH K-L-PTD1. Live S. aureus (˜0.6-1.0 μM diameter) are labeled with green fluorescent wheat germ agglutinin (WGA); PGH K-L-PTD1 is labeled with Alexa Fluor (Red). Yellow staining in the combined panel represents intracellular co-localization (in a single plane as determined by z-stack analysis) of both S. aureus and K-L-PTD1 (blue arrow). FIG. 5e shows confocal microscopy maximum intensity projections (with all z-planes represented) of a MAC-T cell exposed to S. aureus and K-L-PTD1 as in FIG. 5d. The majority of the S. aureus are localized in the thickest part of the cytoplasm surrounding the zone of exclusion created by the nucleus. Many, but not all, S. aureus are co-localized with K-L-PTD1 (yellow) in the combined panel.

FIGS. 6a-6c show that triple-acting fusions can reduce S. aureus-induced mastitis in a mouse model. FIG. 6a depicts the specific activity of peptidoglycan hydrolase fusion constructs in vitro in a turbidity reduction assay with S. aureus Newbould 305 resuspended in lysis buffer to an OD₆₀₀ₙₘ of 1.0 and treated with 1 μM final concentrations of each peptidoglycan hydrolase. The maximum specific activity for three experiments are represented as an average with SEM error bars. FIG. 6b depicts the bacterial burden in mice with mastitis treated with recombinant peptidoglycan hydrolases. Colony Forming Units (CFUs) from infected murine mammary glands following a single treatment with 50 μl of 25 μM peptidoglycan hydrolase (1.25 nmol). Each data point represents the average of duplicate bacterial platings. The horizontal bars represent the average CFU/mg for each group. FIG. 6c illustrates the TNFα response in mice with mastitis that were treated with peptidoglycan hydrolases. Data represents a minimum of 7 measurements in duplicate. Log TNFα was used for statistical analysis. Single asterisks indicate a significant difference from buffer control (p<0.05); double asterisks indicate a significant difference from the parental enzyme (p<0.05).

FIG. 7 depicts a turbidity reduction assay of select peptidoglycan hydrolases against S. aureus Newbould 305. Bacterial cells were resuspended in lysis buffer to an OD₆₀₀ₙₘ of 1.0 and lysed with 1 μM final concentrations of each peptidoglycan hydrolase. The maximum rate of ΔOD of three experiments are represented as an average with error bars representing SEM. Double asterisks indicate significant difference from parental. Mean comparisons were performed with one way ANOVA (P=3.3×10⁻⁵) with t test post hoc analysis with \check{S}idák adjusted alpha (p<0.01).

FIGS. 8a-8d show that triple-acting fusions to PTDs can eradicate intracellular S. aureus in murine osteoblasts. FIG. 8a shows intracellular eradication of Green Fluorescent Protein (GFP)-expressing S. aureus. Neonatal mouse whole calvaria were treated for 4 hours with chimeric peptidoglycan hydrolases or buffer alone, 24 hours post inoculation with GFP-expressing S. aureus. Calvaria were embedded in freeze media, sectioned, and subjected to fluorescence imaging. The image shown is a representative figure for triplicate sections of three separate calvaria. FIG. 8b depicts fluorescence intensity from these sections measured in ImageJ and defined as arbitrary fluorescence standardized to the untreated control. Error bars represent SEM of four replicate experiments. FIG. 8c depicts ex-vivo intracellular S. aureus eradication. Infected calvaria were homogenized and CFU were counted post treatment. Error bars represent SEM. FIG. 8d depicts a murine model of staphylococcal osteomyelitis. C57BL/6J mice were anesthetized and their femurs were surgically exposed. A trough was drilled through the bone cortex and the damaged bone sites were inoculated with 1×10³ CFU S. aureus in agarose beads. After 24 hours, mice were treated (i.m. to site of infection) twice in a 24 h period with PBS or 5 mg/kg of triple fusion K-L or K-L-PTD1. The femurs were removed, homogenized, and plated to quantify the bacterial load. Bars indicate the average CFU recovered (N=6). Both triple fusion K-L (p=0.012) and K-L-PTD1 (p=0.021) significantly reduced bacterial load as compared to no treatment, but the presence or absence of PTD1 had no significant effect (p=0.73). Asterisks represent statistical significance as determined by one-way ANOVA followed by Tukey's posthoc test.

FIG. 9a-9b show that PTD1 enhances triple fusion K-L eradication of dynamic MRSA biofilms. FIG. 9a shows confocal microscopy of biofilm with Live/Dead staining and single 1 μm z-stack images in the middle of NRS382 (USA 100) biofilms treated with 100 μg/ml (1.4 μM) triple fusion K-L, K-L-PTD1, or vancomycin (Van) at a flow rate of 0.5 ml/min for 0, 60, or 120 minutes. Biofilms were pre-stained with the Live/Dead stain (see methods) and viewed with 20× magnification. FIG. 9b depicts bacterial viability. Analysis of bacterial viability in NRS382 dynamic biofilms is based on mean fluorescent intensities of the Live/Dead viability stain when exposed to 100 μg/ml of PGH K-L (1.4 μM), K-L-PTD1 (1.4 μM), or vancomycin (69 μM) at a flow rate of 0.5 ml/min for 2 h and compared to PBS. Error bars represent SEM (N=3) of three independent 100×100 pixel squares identically located in each biofilm z-stack. All values were found to be significantly different using a two-tailed, unpaired, t-test; K-L vs K-LPTD1 (p=0.00064), K-L vs. vancomycin (p=0.00036), and K-L-PTD1 vs. vancomycin (p=0.000023).

FIG. 10 shows that Mupirocin treatment reduces S. aureus nasal colonization in rats. Reduction in nasal colonization was observed when rats were challenged on day 0 with 10⁷ CFU S. aureus strain ALR and then treated twice per day on days 5, 6, and 7 with 10 μl of 2% mupirocin calcium ointment (Bactroban Nasal) or petrolatum/softisan placebo. Noses from rats were homogenized and cultured quantitatively on day 10. Each point represents the CFU recovered from an individual rat, and the data were compiled from two independent experiments. Data were analyzed by the Mann-Whitney test, and horizontal lines represent median values. Mupirocin reduced colonization ˜98% compared to the placebo control.

### DETAILED DESCRIPTION OF THE INVENTION

In response to the critical need for novel antimicrobials with reduced resistance development for treating multi-drug resistant (MDR) S. aureus, we have engineered staphylolytic peptidoglycan hydrolases that degrade Gram-positive peptidoglycan. Many peptidoglycan hydrolases are modular in structure with enzymatic and cell wall binding domains (CBDs) separated by a flexible linker (Nelson et al., supra), allowing for recombinant manipulation and generation of chimeric molecules (Schmelcher et al. 2012, supra; Mao et al. 2013. FEMS Microbiol. Lett. 342:30-36; Becker et al. 2009. Gene 443:32-41). On the premise that a bacterium is unlikely to evade three simultaneous peptidoglycan hydrolase activities, we engineered fusion proteins with three unique lytic activities and determined the impact of these chimeras on resistance development. We further modified these triple-acting fusion peptidoglycan hydrolases with protein transduction domains (PTDs) to facilitate entry into mammalian cells and demonstrated their ability to enhance the eradication of intracellular staphylococci in multiple ex vivo and in vivo models.

Peptidoglycan hydrolases derived from bacteriophage endolysins have the added advantage of co-evolution with the bacteria allowing them to target bonds that the host cell can not readily modify, yielding enzymes that are in theory inherently refractory to resistance development (resistance data for endolysins reviewed in Fischetti, V. A. 2005. Trends Microbiol. 13:491-496). A primary advantage conferred by the peptidoglycan hydrolase attack at the pathogen cell wall is the avoidance of most intracellular resistance mechanisms (e.g. efflux pumps). With multiple peptidoglycan-glycosidase, -endopeptidase, and -amidase domains, there is considerable diversity and often near species-specificity of these activities, ensuring low selective pressure on unrelated, co-resident commensal strains, further reducing the potential for resistance development in non-targeted species. These peptidoglycan hydrolases have additional favorable qualities: non-toxic (Nelson et al. 2001. Proc. Natl. Acad. Sci. U.S.A 98:4107-4112), biodegradable, effective on biofilms (Sass and Bierbaum. 2007. Appl. Environ. Microbiol. 73:347-352) and multi-drug resistant strains (O'Flaherty et al. 2005. J. Bacteriol. 187:7161-7164), and synergistic with antibiotics (Daniel et al. 2010. Antimicrob. Agents Chemother. 54:1603-1612) thereby holding great potential for treating MDR bacteria.

LysK was not as refractory to resistance development (measured by its MIC) as previously reported for Streptococcus and Bacillus endolysins (Fischetti, supra) and a staphylolytic fusion construct (Pastagia et al. 2011. Antimicrob. Agents Chemother. 55:738-744) (in serial dilution plating assays). However, our strategy to reduce resistant strain development by creating triple-acting staphylolytic fusions was successful with very little resistance development for triple fusion L-K in vitro, reflecting at least an order of magnitude improvement over either parental enzyme (alone or in combination). Triple fusion L-K also reduced the bacterial load 5-10 fold better than either parental enzyme in a rat nasal decolonization model, with no in vivo resistance development detected. The observed 98% reduction in nasal bacterial load was virtually identical to the 98% CFU reduction achieved by mupirocin. Despite these successes, as proteins, the peptidoglycan hydrolases must overcome a unique set of therapeutic hurdles.

Peptidoglycan hydrolases are potentially antigenic and engender host immune responses in a manner similar to that seen in phage-based therapies (Gorski et al. 2012. Adv. Virus Res. 83:41-71). However, peptidoglycan hydrolases show minimal immunogenicity in mammals, and adverse responses have not been reported. Bovine intramammary infusions of lysostaphin resulted in detectable levels of specific antibodies only after 18-21 treatments. The antibodies were not neutralizing, nor did they elicit observable effects on the host animal or eliminate the antimicrobial properties of lysostaphin (Daley and Oldham. 1992. Vet. Immunol. Immunopathol. 31:301-312). Serum antibodies raised to phage endolysins specific to Bacillus anthracis, Streptococcus pyogenes, or Streptococcus pneumoniae slowed, but did not inhibit microbial killing in vitro (Fischetti, supra; Loeffler and Fischetti. 2003. Antimicrob. Agents Chemother. 47:375-377). There is also a concern for proinflammatory components released from lysed bacteria (Borysowski et al. 2006. Exp. Biol. Med. (Maywood) 231:366-377). However, adverse immune responses have not been observed in mouse models for an array of systemically delivered staphylolytic enzyme constructs. In fact, seven out of nine endolysins provided 100% protection from MRSA bacteremia versus 20% survival at 48 hours post infection in buffer or oxacillin treated animals. Furthermore, a reduced TNFα response (to near baseline) when mice with mastitis were treated with our triple-acting fusion peptidoglycan hydrolases indicates a reduced inflammatory response compared to untreated controls.

A separate therapeutic hurdle is created by systemic infections of S. aureus during which the pathogen can evade the host immune system (and most antibiotics) through intracellular localization and sequestration of the pathogen. Intracellular invasion has been reported for bovine mastitis [where S. aureus has been identified within mammary alveolar cells and macrophages isolated from milk (Hebert et al., supra; Peton and Le Loir, supra)] and is implicated by the high frequency of MDR S. aureus in recurring osteomyelitis in “cured” blast wound victims from Middle Eastern war zones (Yun et al., supra). Toxic levels of conventional antibiotics are often required to treat classic intracellular pathogens (Gaspar et al. 2008. Curr. Top. Med. Chem. 8:579-591). To address this concern, we engineered our peptidoglycan hydrolase constructs with 11 different protein transduction domains (PTDs) and identified the optimal PTD domain(s) to facilitate import of the peptidoglycan hydrolases into cultured cells. Initial data with lysostaphin indicated that a PTD was essential for eradication of intracellular S. aureus within both the cultured bovine mammary epithelial cell line (MAC-T, Nexia Biotechnologies, Quebec, Canada) and human brain microvascular endothelial cells (hBMECs). In contrast, triple fusion K-L did not require a PTD to invade cultured bovine mammary cells or murine osteoblasts and showed a similar efficacy with or without a PTD in bone infection models. Both LysK and triple fusion L-K were ineffective at intracellular eradication when fused to any of the eleven PTDs. Despite equivalent intracellular efficacy with either triple fusion K-L or construct K-L-PTD1 in cultured mammary cells, the latter construct showed almost 3 orders of magnitude greater CFU reduction in vivo in the mastitis model. These inconsistencies underline the complexity of PTDs, cargo proteins, and cellular uptake mechanisms to achieve intracellular localization as well as the vagaries of ex vivo versus in vivo experiments (e.g. physiology of infection; monoculture vs. live tissue). Triple fusions K-L and L-K both harbor virtually identical sequences, with only the order of the domains being rearranged, suggesting that an alternative tertiary structure likely contributes to their differential efficacy.

The ability of peptidoglycan hydrolases to reduce or eradicate static biofilms (Sass and Bierbaum. 2007. Appl. Environ. Microbiol. 73:347-352) is an important advantage over conventional antibiotics, since biofilms are proposed to play a critical role in infectious disease (Biel, M. A. 2010. Methods Mol. Biol. 635:175-194). There was an apparent benefit conferred by adding a PTD to the engineered triple fusion K-L in our dynamic S. aureus biofilm eradication experiments.

The ability to engineer a peptidoglycan hydrolase antimicrobial with qualities not readily achievable with conventional antibiotics (refractory to resistance development, biofilm eradication, treatment of intracellular and MDR pathogens) offers the possibility that other desirable traits could be engineered into peptidoglycan hydrolase antimicrobials. Engineered peptidoglycan hydrolases comprise a much needed class of antibacterials for treating multiple-drug resistant pathogens.

According to the present invention, the terms “nucleic acid molecule”, “nucleic acid sequence”, “polynucleotide”, “polynucleotide sequence”, “nucleic acid fragment”, “isolated nucleic acid fragment” are used interchangeably herein. These terms encompass nucleotide sequences and the like. A polynucleotide may be a polymer of RNA or DNA that is single- or double-stranded and that optionally contains synthetic, non-natural or altered nucleotide bases. A polynucleotide in the form of a polymer of DNA may be comprised of one or more segments of cDNA, genomic DNA, synthetic DNA, or mixtures thereof. This will also include a DNA sequence for which the codons encoding, for example, the LysK-lysostaphin fusion protein according to the invention will have been optimized according to the host organism in which it will be expressed, these optimization methods being well known to those skilled in the art.

The term “isolated” polynucleotide refers to a polynucleotide that is substantially free from other nucleic acid sequences, such as other chromosomal and extrachromosomal DNA and RNA, that normally accompany or interact with it as found in its naturally occurring environment. However, isolated polynucleotides may contain polynucleotide sequences which may have originally existed as extrachromosomal DNA but exist as a nucleotide insertion within the isolated polynucleotide. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “construct” refers to a recombinant nucleic acid, generally recombinant DNA, that has been generated for the purpose of the expression of a specific nucleotide sequence(s), or is to be used in the construction of other recombinant nucleotide sequences. A “construct” or “chimeric gene construct” refers to a nucleic acid sequence encoding a protein, operably linked to a promoter and/or other regulatory sequences.

The term “operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter) or a DNA sequence and a regulatory sequence(s) are connected in such a way as to permit gene expression when the appropriate molecules (e.g., transcriptional activator proteins) are bound to the regulatory sequence(s).

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence that can stimulate promoter activity and may be an innate element of the promoter or a heterologous element inserted to enhance the level or tissue-specificity of a promoter.

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein. “cDNA” refers to a DNA that is complementary to and derived from an mRNA template.

As used herein, “recombinant” refers to a nucleic acid molecule which has been obtained by manipulation of genetic material using restriction enzymes, ligases, and similar genetic engineering techniques as described by, for example, Sambrook et al. 1989. Molecular Cloning: A Laboratory Manual, Second Edition, Cold Spring Harbor Laboratory Press, Cold Spring Harbor, N.Y. or DNA Cloning: A Practical Approach, Vol. I and II (Ed. D. N. Glover), IRL Press, Oxford, 1985. “Recombinant,” as used herein, does not refer to naturally occurring genetic recombinations.

As used herein, the terms “encoding”, “coding”, or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

A “protein” or “polypeptide” is a chain of amino acids arranged in a specific order determined by the coding sequence in a polynucleotide encoding the polypeptide. Each protein or polypeptide has a unique function.

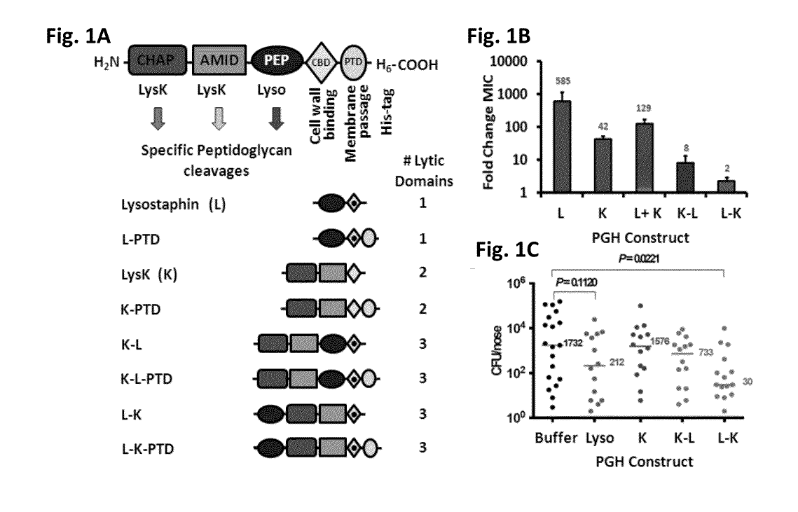

The invention includes, as depicted in FIG. 1a, functional LysK-lysostaphin-cell wall binding domain (Lysk-Lyso-CBD) fusion protein, triple fusion K-L; LysK-lysostaphin-cell wall binding domain-protein transduction domain (Lysk-Lyso-CBD-PTD) fusion protein, K-L-PTD; Lyso-LysK-CBD fusion protein with rearranged domain order, triple fusion L-K; and Lyso-Lysk-CBD-PTD, L-K-PTD; and functional fragments thereof, as well as mutants and variants having the same biological function or activity. As used herein, the terms “functional fragment”, “mutant” and “variant” refers to a polypeptide which possesses biological function or activity identified through a defined functional assay and associated with a particular biologic, morphologic, or phenotypic alteration in the cell. The term “functional fragments” refers to all fragments of the lytic domains of the triple fusion polypeptide of the invention that retain lytic activity and function to lyse staphylococcal bacteria.

The characteristic of the three different peptidoglycan hydrolase domains described as “specifically targets and attacks” means that each of the three different domains cuts the peptidoglycan cell wall at a different, unique covalent bond of the peptidoglycan cell wall of live, untreated S. aureus from without.

Modifications of the primary amino acid sequence of the lytic domains of the invention may result in further mutant or variant proteins having substantially equivalent activity to the fusion polypeptides described herein. Such modifications may be deliberate, as by site-directed mutagenesis, or may occur by spontaneous changes in amino acid sequences where these changes produce modified polypeptides having substantially equivalent activity to the endolysin polypeptides of the triple fusion polypeptide. Any polypeptides produced by minor modifications of the endolysin primary amino acid sequence are included herein as long as the biological activity endolysin is present; e.g., having a role in pathways leading to lysis of staphylococcal bacteria.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to modifications of the nucleic acid fragments of the instant invention such as deletion or insertion of nucleotides that do not substantially affect the functional properties of the resulting transcript. It is therefore understood that the invention encompasses more than the specific exemplary nucleotide or amino acid sequences and includes functional equivalents thereof. Alterations in a nucleic acid fragment that result in the production of a chemically equivalent amino acid at a given site, but do not affect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (1985. Nucleic Acid Hybridization, Hames and Higgins, Eds., IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. An indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Thus, isolated sequences that encode a LysK-Lyso-CBD and/or Lyso-Lysk-CBD fusion polypeptide sequences disclosed herein, or to fragments thereof, are encompassed by the present invention.

Substantially similar nucleic acid fragments of the instant invention may also be characterized by the percent identity of the amino acid sequences that they encode to the amino acid sequences disclosed herein, as determined by algorithms commonly employed by those skilled in this art. Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988. CABIOS 4:11-17), the local homology algorithm of Smith et al. (1981. Adv. Appl. Math. 2:482); the homology alignment algorithm of Needleman and Wunsch (1970. J. Mol. Biol. 48:443-453); the search-for-similarity-method of Pearson and Lipman (1988. Proc. Natl. Acad. Sci. 85:2444-2448; the algorithm of Karlin and Altschul (1990. Proc. Natl. Acad. Sci. USA 87:2264), modified as in Karlin and Altschul (1993. Proc. Natl. Acad. Sci. USA 90:5873-5877).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “substantial identity” of polynucleotide sequences means that a polynucleotide comprises a sequence that has at least 80% sequence identity, preferably at least 85%, more preferably at least 90%, most preferably at least 95% sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 80%, preferably at least 85%, more preferably at least 90%, and most preferably at least 95%. Preferably, optimal alignment is conducted using the homology alignment algorithm of Needleman et al. (1970. J. Mol. Biol. 48:443).

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST. In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification and isolation. In addition, short oligonucleotides of 12 or more nucleotides may be use as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise a particular plant protein. The skilled artisan, having the benefit of the sequences as reported herein, may now use all or a substantial portion of the disclosed sequences for purposes known to those skilled in this art. Thus, such a portion represents a “substantial portion” and can be used to establish “substantial identity”, i.e., sequence identity of at least 80%, compared to the reference sequence. Accordingly, the instant invention comprises the complete sequences as reported in the accompanying Sequence Listing, as well as substantial portions at those sequences as defined above.

Fragments and variants of the disclosed nucleotide sequences and proteins encoded thereby are also encompassed by the present invention. By “fragment” a portion of the nucleotide sequence or a portion of the amino acid sequence and hence protein encoded thereby is intended. Fragments of a nucleotide sequence may encode protein fragments that retain the biological activity of the native protein and hence have LysK-Lyso-CBD fusion polypeptide- and/or Lyso-LysK-CBD fusion polypeptide-like activity. Alternatively, fragments of a nucleotide sequence that are useful as hybridization probes may not encode fragment proteins retaining biological activity.

By “variants” substantially similar sequences are intended. For nucleotide sequences, conservative variants include those sequences that, because of the degeneracy of the genetic code, encode the amino acid sequence of one of the LysK-lysostaphin fusion polypeptides, LysK-Lyso-CBD fusion polypeptide- and/or Lyso-LysK-CBD fusion polypeptide of the invention. Naturally occurring allelic variants such as these can be identified with the use of well-known molecular biology techniques, as, for example, with polymerase chain reaction (PCR), a technique used for the amplification of specific DNA segments. Generally, variants of a particular nucleotide sequence of the invention will have generally at least about 90%, preferably at least about 95% and more preferably at least about 98% sequence identity to that particular nucleotide sequence as determined by sequence alignment programs described elsewhere herein.

By “variant protein” a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein is intended. Variant proteins encompassed by the present invention are biologically active, that is they possess the desired biological activity, that is, LysK-Lyso-CBD fusion polypeptide and/or Lyso-LysK-CBD fusion polypeptide protein activity as described herein. Such variants may result from, for example, genetic polymorphism or from human manipulation. Biologically active variants of a LysK-Lyso-CBD fusion polypeptide and/or Lyso-LysK-CBD fusion polypeptide of the invention will have at least about 90%, preferably at least about 95%, and more preferably at least about 98% sequence identity to the amino acid sequence for the protein of the invention as determined by sequence alignment programs described elsewhere herein. A biologically active variant of a protein of the invention may differ from that protein by as few as 1-15 amino acid residues, or even 1 amino acid residue.

The polypeptides of the invention may be altered in various ways including amino acid substitutions, deletions, truncations, and insertions. Novel proteins having properties of interest may be created by combining elements and fragments of proteins of the present invention, as well as with other proteins. Methods for such manipulations are generally known in the art. Thus, the genes and nucleotide sequences of the invention include both the naturally occurring sequences as well as mutant forms. Likewise, the proteins of the invention encompass naturally occurring proteins as well as variations and modified forms thereof. Such variants will continue to possess the desired LysK-Lyso-CBD fusion polypeptide and/or Lyso-LysK-CBD fusion polypeptide activity. Obviously, the mutations that will be made in the DNA encoding the variant must not place the sequence out of reading frame and preferably will not create complementary regions that could produce secondary mRNA structure.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays where the effects of LysK-Lyso-CBD fusion polypeptide and/or Lyso-LysK-CBD fusion polypeptide can be observed.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein.

The staphylococcal control compositions of the invention comprise the antimicrobial composition of the invention dissolved or suspended in an aqueous carrier or medium. The composition may further generally comprise an acidulant or admixture, a rheology modifier or admixture, a film-forming agent or admixture, a buffer system, a hydrotrope or admixture, an emollient or admixture, a surfactant or surfactant admixture, a chromophore or colorant, and optional adjuvants. The preferred compositions of this invention comprise ingredients which are generally regarded as safe, and are not of themselves or in admixture incompatible with milk or milk by-products or human and veterinary applications. Likewise, ingredients may be selected for any given composition which are cooperative in their combined effects whether incorporated for antimicrobial efficacy, physical integrity of the formulation or to facilitate healing and health in medical and veterinary applications, including for example in the case of mastitis, healing and health of the teat or other human or animal body part. Generally, the composition comprises a carrier which functions to dilute the active ingredients and facilitates stability and application to the intended surface. The carrier is generally an aqueous medium such as water, or an organic liquid such as an oil, a surfactant, an alcohol, an ester, an ether, or an organic or aqueous mixture of any of these, or attached to a solid stratum such as colloidal gold. Water is preferred as a carrier or diluent in compositions of this invention because of its universal availability and unquestionable economic advantages over other liquid diluents.

Avoiding the generalized use of broad range antimicrobials and using highly specific antimicrobials for just the target organisms involved, should help reduce the ever-increasing incidence of antibiotic resistance.

### EXAMPLES

Having now generally described this invention, the same will be better understood by reference to certain specific examples, which are included herein only to further illustrate the invention and are not intended to limit the scope of the invention as defined by the claims.

### Example 1

### Bacterial Strains

Mastitis isolates S. aureus Newbould 305, Staphylococcus hyicus MP01, Staphylococcus chromogenes MP02, Staphylococcus simulans MP03, Staphylococcus epidermidis MP04, Staphylococcus xylosus MP05, and Staphylococcus warneri MP06 were gifts from Max Paape (USDA, Beltsville). Strain S. aureus SA113 was provided by Andreas Peschel (University of Tübingen, Germany). S. aureus strains Newman (Regev-Yochay et al. 2006. J. Bacteriol. 188:4996-5001); ALR (Regev-Yochay et al., supra; UAMS-1 (Marriott et al. 2005. Bone 37:504-512) and ISP479C (Sheen et al. 2010. J. Mol. Med. (Berl). 88:633-639). (UAMS-1 and ISP479C were described previously.) The remaining strains (designated with NRS numbers) were obtained from Network on Antimicrobial Resistance in S. aureus (NARSA) repository. The antibiotic resistance profiles of the strains are listed in Table 1.

TABLE 1
Minimum Inhibitory Concentration of Constructs against S. aureus.

| | | | Minimum Inhibitory Conc. | | | |
|---|---|---|---|---|---|---|
| | | | (median μg/ml) | | | |
| Strain⁺ | Resistant^{#} | Intermediate | L* | K* | K-L* | L-K* |
| S. aureus Newman | | | 0.77 | 47 | 7.0 | 7.8 |
| S. aureus ALR | S | | 0.77 | 34 | 7.0 | 4.4 |
| MARSA Strains | | | | | | |
| USA 100 (NRS 382) | Cp Cc E Ox P | | 1.2 | 96 | 14 | 20 |
| USA 200 (NRS 383) | Cp Cc E Gm Ox P | | 1.2 | 34 | 5.5 | 5.9 |
| USA 300 (NRS 384) | E Ox P | Cp | 1.2 | 75 | 11 | 20 |
| USA 400 (NRS 123) | Ox P Te | | 1.2 | 75 | 7.0 | 16 |
| USA 500 (NRS 385) | Cp Cc E Gm Ox P | | 1.2 | 67 | 7.0 | 12 |
| | Te SXT | | | | | |
| USA 600 (NRS 22) | Cp Gm Ox P SXT | Va (Etest) | 1.2 | 75 | 7.0 | 6.8 |
| N315 (NRS 70) | Cc E Ox P | | 0.68 | 37 | 7.0 | 16 |
| Sanger 252 (NRS 71) | Cp Cc E Ox P | | 0.58 | 29 | 5.5 | 6.8 |
| NRS 192 | Ox P | E | 1.2 | 80 | 11 | 16 |
| NRS 193 | Ox P | | 1.2 | 37 | 7.0 | 12 |
| NRS 194 | Ox P | | 1.2 | 69 | 14 | 12 |
| NRS 209 | Ox P | | 0.97 | 40 | 7.0 | 12 |
| NRS 271 | Cp Lz Ox P | E | 0.58 | 44 | 7.0 | 9.8 |
| Mastitis | | | | | | |
| S. aureus Newbould 305 | | | 12 | 34 | 11 | 12 |
| S. chromogenes MP02 | | | 0.58 | 16 | 3.5 | 0.37 |
| S. epidermidis MP04 | | | 6.2 | 8.4 | 3.5 | 4.2 |
| S. hyicus MP01 | | | 0.29 | 8.4 | 1.8 | 0.37 |
| S. simulans MP03 | | | 1.5 | 20 | 3.5 | 5.9 |
| S. warneri MP06 | | | 1.5 | 28 | 5.3 | 1.5 |
| S. xylosus MP05 | | | 0.39 | 8.4 | 3.5 | 4.0 |

*All Constructs are described in FIG. 1A.
⁺All NRS strains were obtained from the Network on Antimicrobial Resistance in Staphylococcus aureus (NARSA) collections (Retrieved from the Internet: <URL: niaid.nih.gov/labsandresources/resources/dmid/narsa/Pages/default.aspx).
^{#}Ciprofloxacin (Cp), Clindamycin (Cc), Erythromycin (E), Gentamicin (Gm), Linezolid (Lz), Oxacillin (OX), Penicillin (p), Streptomycin (S), Tetracycline (Te), Trimethoprimsulfamethoxaxole (SXT), Vancomycin (Va).

### Example 2

### Peptidoglycan Hydrolase Expression Constructs; Engineering Triple-Acting Peptidoglycan Hydrolase Fusions

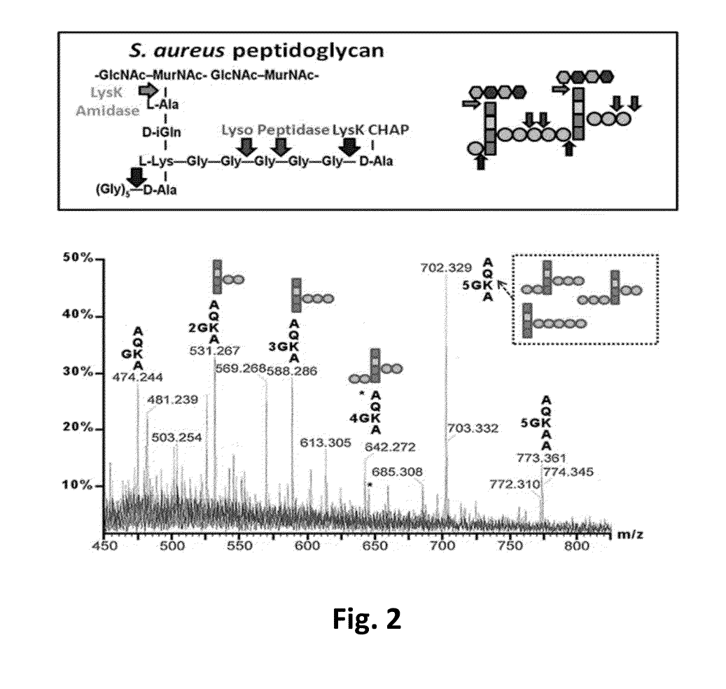

Peptidoglycan hydrolases harbor one or two lytic domains along with one or more cell wall binding domains. To impede resistance development, we engineered peptidoglycan hydrolase antimicrobial fusion proteins to harbor three unique lytic activities derived from two parental peptidoglycan hydrolases, lysostaphin, a staphylolytic bacteriocin (Browder et al. 1965. Biochem. Biophys. Res. Commun. 19:389) and LysK, a staphylococcal bacteriophage K endolysin (O'Flaherty et al. 2005. J. Bacteriol. 187:7161-7164)(FIG. 1a). Lysostaphin contains a glycyl-glycine M23 endopeptidase domain (Pattee, P. A. 1981. J. Bacteriol. 145:479-488. LysK contains two catalytic domains, an N-terminal cysteine, histidine-dependent amidohydrolase/peptidase (CHAP endopeptidase) and an N-acetylmuramoyl-L-alanine amidase (Schaffer et al. 2006. Infect. Immun. 74:2145-2153; Schmelcher et al. 2012. Appl. Environ. Microbiol. 78:2297-2305). Both parental enzymes contain a similar C-terminal SH3b cell wall binding domains. LysK and lysostaphin are active against antibiotic-sensitive and antibiotic-resistant S. aureus strains (Table 1), and in combination demonstrate synergy in killing S. aureus cells (Donovan et al. 2009, supra). Each enzymatic domain targets separate, unique bonds in the S. aureus peptidoglycan (Rigden et al. 2003, supra) (illustrated in FIG. 2) making them ideal candidates for inclusion in our triple-acting fusions.

A plasmid harboring the gene encoding the 247 amino acid mature (secreted) lysostaphin (Construct L) (UniProtKB/Swiss-Prot #: P10547.2) was a gift from David Kerr, University of Vermont. The staphylococcal phage K endolysin cDNA encoding the 495 amino-acid lysK gene product (Construct K) (Genbank AAO47477.2) was provided by Paul Ross (O'Flaherty et al. 2005. J. Bacteriol. 187:7161-7164). All constructs were performed in E. coli DH5α (Invitrogen, Carlsbad, Calif.), via PCR cloning (Becker et al. 2008, supra), or conventional DNA fragment isolation and ligation using standard methods, and were verified by DNA sequencing. All constructs harbor a C-terminal His₆ tag derived from pET21a (EMD Biosciences, San Diego, Calif.). All PCR primers and plasmids are listed in Table 2.

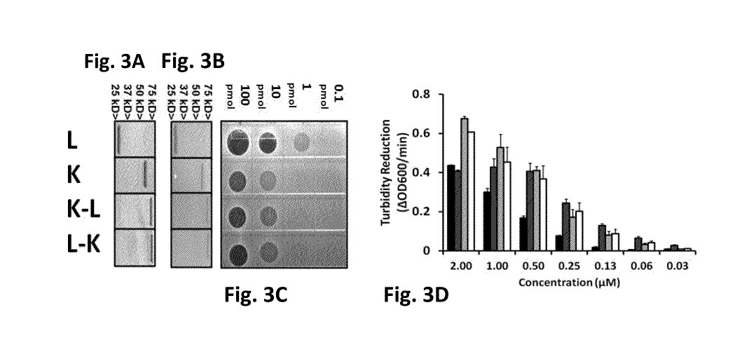

We previously described a head-to-tail fusion of LysK-lysostaphin that included two cell wall binding domains (Donovan et al. 2009, supra) and showed weak staphylolytic activity (Rigden et al. 2003, supra). Improvements were made to create two triple acting constructs, each with a single cell wall binding domain (the SH3b domain from lysostaphin). The first triple-acting peptidoglycan hydrolase (K-L) was constructed with both LysK lytic domains fused to the N-terminus of lysostaphin. The second triple-acting construct (L-K) inserted the LysK lytic domains between lysostaphin's M23 endopeptidase domain and SH3b cell wall binding domain (FIG. 1a). Parental and triple-acting fusion proteins were expressed in E. coli, and purified by nickel chromatography via a C-terminus His₆ tag (˜98% pure in SDS PAGE) yielding a single band in zymogram analysis (FIG. 3a-3b).

TABLE 2
Vectors and DNA Primer Sequences.

| | | SEQ |
|---|---|---|
| PRIMER | SEQUENCE | ID NO: |
| LysoAA1NdeIF | 5′-ACGTACGTCATATGGCTG | 73 |
| | CAACACATGAACATTCAGCAC | |
| LysoXhoIR | 5′-GCGCTACTCGAGACCACC | 74 |
| | TGCTTTTCCATATC | |
| LysoSalIF | 5′-ATCATCGTCGACGCTGCA | 75 |
| | ACACATGAACATTCAGCAC | |
| LysoAD155XhoIR | 5′-GTTTGTCTCGAGACCTGT | 76 |
| | ATTCGG | |
| LysoSH3bSalIF | 5′-GCGCATCTCGAGACAGTA | 77 |
| | ACTCCAACGCCG | |
| pET21aStyIR | 5′-CGTTTAGAGGCCCCAAGG | 78 |
| | GGTTATG | |
| LysKaa1SalF | 5′-GATATAGTCGACGCTAAG | 79 |
| | ACTC | |
| PLASMID | PROTEIN EXPRESSED | |
| p5301 | Lysostaphin-His₆ | |
| p3514 | LysK-His₆ | |
| pSB1101 | Triple Fusion K-L-His₆ | |
| pSB1801 | Triple Fusion L-K-His₆ | |

Underline = Restriction enzyme recognition sequence

The cDNA molecules encoding the triple fusion polypeptides K-L, L-K and the triple fusion PTD polypeptides, K-L-PTD1 comprise nucleotides encoding C-terminal LE,HHHHHH residues and are identified by SEQ ID NOs: 5, 7 and 53, respectively. The proteins encoded by these nucleic acid sequences are identified by SEQ ID NOs: 6, 8 and 54, respectively. The expressed proteins are His-tagged with eight additional amino acid residues introduced at the C-terminus corresponding to the XhoI site (Leu-Glu) followed by six His residues.

Triple fusion K-L encoded by expression vector pSB1101 was created from the lysK truncation LysK390′ expression vector pSB0301, previously described (Becker et al. 2009, supra). The lysostaphin fragment fused to the 3′ end of pSB1101 was amplified with primers Lyso SalIF and LysoXhoIR, digested with XhoI and SalI, and introduced into pSB0301 linearized at the XhoI site, generating pSB1101.

Triple fusion L-K encoded by expression vector pSB1801 was created in several steps. The lysostaphin gene from plasmid p5301 was truncated by PCR-amplifying the M23 peptidase domain with the primers LysoAD155XhoIR and LysoAA1 NdeI F and introducing this PCR product into NdeI+XhoI-digested pET21a, thereby generating pSB1701. A second intermediate construct was produced by amplification of the lysostaphin SH3b domain from plasmid template p5301 with the primers LysoSH3b SalIF and pET21a StyIR, digesting with SalI+StyI, and introducing the amplified fragment into XhoI+StyI-digested pSB0301, thereby generating plasmid pSB1001. The final triple fusion L-K construct was generated by introducing the PCR product generated by amplification of the template pSB1001 with the primers LysKaaSaIF and pET21aStyIR (harboring the two lytic domains of LysK) into XhoI+StyI-digested pSB1701, thereby generating pSB1801.

Fusion of peptidoglycan hydrolase to PTD sequences was accomplished by reverse translation of the 12 PTD sequences (Table 3) into nucleotide sequences (with an E. coli codon usage bias), followed by commercial synthesis (Genscript, Piscataway, N.J.). The individual sequences were inserted at the XhoI site of pET21a such that each PTD coding sequence was in frame with the His₆ coding sequences of the vector. The four peptidoglycan hydrolase sequences (encoding mature lysostaphin, lysK, and triple fusions K-L and L-K) were introduced into these PTD-His₆ encoding vectors via standard procedures following restriction enzyme digests of the parental peptidoglycan hydrolase vectors (described above) at unique sites (XbaI or NdeI and XhoI) to generate DNA fragments harboring the entire peptidoglycan hydrolase coding region, and ligation of these fragments into similarly digested pET21a vectors harboring the 12 individual PTD sequences.

TABLE 3
Protein Transduction Domains (PTD)

| | | SEQ | |
|---|---|---|---|
| PTD | SEQUENCE | ID NO: | REF.^{#} |
| PTD 1 | RQIKIWFQ | 80 | 35 |
| (Antp 43-58) | NRRMKWKK | | |
| PTD 2 | WEEAKLAK | 81 | 36 |
| (Kala- Synthetic) | ALAKALAK | | |
| | HLAKALAK | | |
| | ALKACEA | | |
| PTD 3 | MVTVLFRR | 82 | 37 |
| (M918- Synthetic) | LRIRRASG | | |
| | PPRVRV | | |
| PTD 4 | KLALKLAL | 83 | 38 |
| (MAP- Synthetic) | KALKAALK | | |
| | LA | | |
| PTD 6 | RRQRRTSK | 84 | 39 |
| (PTD5- Synthetic) | LMKR | | |
| PTD 7 | LLIILRRR | 85 | 40 |
| (pVEC 615-632) | IRKQAHAH | | |
| | SK | | |
| PTD 8 | RRRRRRRR | 86 | 41 |
| (Poly Arg 8- | | | |
| Synthetic | | | |
| PTD 9 | GRKKRRQR | 87 | 42 |
| (TAT 48-60) | RRPPQ | | |
| PTD 10 | GWTLNSAG | 88 | 43 |
| (Transportan- | YLLGKINL | | |
| Chimeric) | KALAALAK | | |
| | KIL | | |
| PTD 11 | AGYLLGKI | 89 | 44 |
| (Transportan 10- | NLKALAAL | | |
| Chimeric | AKKIL | | |
| PTD 12 | RKKRRQRR | 90 | 45 |
| (TAT 49-57) | R | | |

^{#}35. Derossi et al. 1996. J Biol. Chem. 271:18188-18193
36. Wyman et al. 1997. Biochemistry 36:3008-3017
37. El-Andaloussi et al. 2007. Mol. Ther. 15:1820-1826
38. Scheller et al. 1999. J. Pept. Sci. 5:185-194
39. Mi et al. 2000. Mol. Ther. 2:339-347
40. Elmquist et al. 2001. Exp. Cell Res. 269:237-244
41. Wender et al. 2000. Proc. Nat.Acad. Sci. U. S. A. 97:13003-13008
42. Vives et al. 1997. J. Biol. Chem. 272:16010-16017
43. Pooga et al. 1998. FASEB J. 12:67-77
44. Soomets et al. 2000. Biochim. Biophys. Acta. 1467:165-176

### Example 3

### Protein Expression and Purification

Recombinant peptidoglycan hydrolases were expressed in E. coli BL21 (DE3) (Novagen) grown to mid-log phase at 37° C. in modified LB broth (15 g tryptone, 8 g yeast extract, 5 g NaCl per liter, pH 7.8) with shaking, induced with 1 mM IPTG, and expressed for 20 h at 10° C. Cells were disrupted by sonication and purified via nickel affinity chromatography (Ni-NTA-agarose), per the manufacturer's instructions (Qiagen, Carlsbad Calif.) to >95% purity as described previously (Becker et al. 2008, supra). For peptidoglycan hydrolases used in animal and cell culture experiments, endotoxin was removed via Triton X-114 washes of protein on Ni-NTA columns (Reichelt et al. 2006. Protein Expr. Purif. 46:483-488). Representative random samples were evaluated for endotoxin content via the Limulus amoebocyte lysate assay (LAL QCL-1000, Lonza, Walkersville, Md.) and shown to be <5 Units/ml (e.g., for nasal colonization <5 Units/˜40 mg protein) endotoxin after purification.

### Example 4

### Preliminary Characterization of Peptidoglycan Hydrolase Activity

SDS-PAGE, zymogram, turbidity reduction and plate lysis assays were performed as described previously (Becker et al. 2009, supra) with minor modifications as follows. For SDS-PAGE and zymogram analysis, one μg of the purified peptidoglycan hydrolase proteins and Kaleidoscope protein standards (Invitrogen, Carlsbad, Calif.) were analyzed by 15% SDS-PAGE. SDS-PAGE and zymogram gels were prepared and electrophoresed in parallel. Zymograms incorporated embedded cells equivalent to 300 ml of mid-logarithmic phase (OD₆₀₀ₙₘ 0.4-0.6) S. aureus Newman into the SDS-PAGE matrix. The SDS-PAGE gels were stained with Coomassie blue using standard protocols, and zymograms were washed twice in excess water for 30 min to remove SDS and incubated for <1 h at room temperature in water until cleared zones developed.

For the plate lysis assays, purified enzymes were serially diluted in saline lysis buffer (SLB; 150 mM NaCl 10 mM Tris buffer, pH 7.5) with 15% glycerol, to yield concentrations of 100, 10, 1, and 0.1 pmoles/10 μl. S. aureus Newman was cultivated in tryptic soy broth (TSB) to an OD_{600 nm}=0.4-0.6. The bacterial cells were harvested and diluted to yield a suspension with an OD_{600 nm} of 0.1. Tryptic soy agar (TSA) plates were flooded with 3 ml of the bacterial cell suspension. Excess culture was removed, and the plates were air dried at room temperature for ˜30 min in a laminar flow hood. 10 μl of each peptidoglycan hydrolase dilution was then spotted onto the air-dried lawn, allowed to air dry, and the culture plates incubated overnight at 37° C. The following day, plates were evaluated visually and photographed.

The Minimum Inhibitory Concentrations (MIC) of each protein for multiple S. aureus strains was determined as previously described (Jones et al. 1985. In: Manual of Clinical Microbiology, Eds. Balows et al. American Society of Microbiology, Washington D.C., pp. 972-977) with the following modifications. Enzymes were serially diluted two fold across a 96 well plate, such that after dilution, 50 μl of the enzyme solution in 300 mM NaCl, 50 mM NaH₂PO₄, 30% glycerol pH 7.5, remained in each well. To each well was added 50 μl of TSB and 100 μl of S. aureus Newman in TSB (diluted to 5×10⁶ cells/ml). The plates were incubated 20 h at 37° C. and read with a 96 well plate reader. MIC determination values were reported as the median of ≧4 replicates (Table 1).

Three lytic peptidoglycan hydrolase enzyme activities were confirmed. LysK is a 495 amino acid protein with a C-terminal SH3b CBD (SH3-5, Pfam PF08460) (Finn et al. 2008. Nucleic Acids Res. 36: D281-D288) and two lytic domains (FIG. 1a). The N-terminal lytic domain is a cysteine, histidine-dependent amidohydrolase/peptidase (CHAP) domain (Rigden et al. 2003. Trends Biochem. Sci. 28:230-234) (Pfam PF05257), and the second (internal) lytic domain has been classified as an amidase-2 domain (PFAM PF01510). Both lytic domains are active and show specific cleavage sites on purified S. aureus peptidoglycan (Becker et al. 2009, supra; Donovan et al. 2009, supra). The CHAP endopeptidase activity cleaves between the D-alanine of the stem peptide and the first glycine of the pentaglycine cross-bridge peptide, and the amidase-2 domain harbors an N-acetylmuramoyl-L-alanine amidase activity that cleaves between the N-acetylmuramic acid of the polysaccharide strand and L-alanine of the stem peptide (FIG. 2). Mature lysostaphin is a 246 amino acid protein with a single enzymatic domain (FIG. 1a), an N-terminal M23 glycyl-glycine endopeptidase (Pfam PF01551) that cleaves between the second and third, or third and fourth, glycines of the S. aureus pentaglycine cross bridge (FIG. 2) (Browder et al., supra) Like many staphylococcal peptidoglycan hydrolase and phage endolysins (Becker et al. 2009, supra), the bacteriocin lysostaphin also harbors a C-terminal SH3b cell wall binding domain (SH3-5, Pfam PF08460).

Purified S. aureus peptidoglycan was digested by triple fusions created with LysK and lysostaphin and the products examined via Electrospray Ionization Mass Spectrometry (ESI-MS). For all triple fusion constructs examined, the peptidoglycan digestion products yielded peaks at m/z 702, 645, 588, 531, and 474 that are identical to the peaks obtained with a double digestion with both enzymes (including triple fusions described previously (Donovan et al. 2009, supra). Representative peaks are shown in FIG. 2 for triple fusion K-L. Peaks observed at m/z=588 (A₂QKG₃) and 531 (A₂QKG₂) require cleavage by all three domains. A previously reported head to tail of full length LysK and lysostaphin (with reduced activity compared to triple fusion K-L and L-K) was also reported to maintain all three peptidoglycan cut sites (Donovan et al. 2009, supra).

The LysK-lysostaphin triple-acting peptidoglycan hydrolases demonstrated intermediate activity compared to the parental peptidoglycan hydrolases in both plate lysis (FIG. 3) and minimum inhibitory concentration (MIC) assays (Table 1). Both chimeric peptidoglycan hydrolases (triple fusions K-L and L-K) were as effective as the parental peptidoglycan hydrolases in turbidity reduction assays (FIG. 3d). A reduction in turbidity equates to reduced bacterial viability (Nelson et al., supra). Importantly, all three lytic domains were active in the final fusion constructs as determined by electron spray ionization mass spectrometry on peptidoglycan digestion products (FIG. 2).

### Example 5

### Resistance Development of Triple-Acting Fusion Enzymes

For the MIC repeated exposure method, MIC determinations were performed with S. aureus strain Newman. 100 μl of S. aureus strain Newman culture that survived at ½ the MIC (the first well with visible growth) for each round was inoculated into 5 ml TSB and cultivated to mid-log phase growth. This culture was used as the inoculum for the next round of MIC exposure (overnight growth), and the cycle was repeated for 10 rounds. Cells recovered from the well that represented the ½ MIC concentration on round 10 were confirmed to be S. aureus by PCR (Martineau et al. 1998. J. Clin. Microbiol. 36:618-623) (data not shown).

In the Plate lysis method, bacterial cells were scraped from a sub-lethal (not fully cleared) spot from a plate lysis assay (described above), and these ‘exposed’ cells were used to inoculate 5 ml of TSB and grown for 4-6 hours to generate a new culture and lawn for subsequent exposure. Bacteria were passaged in this assay for up to 10 consecutive days, at which time the S. aureus were tested in MIC assays. Plate lysis repeated exposure experiments were performed in duplicate.

Development of resistance to the triple-acting fusion enzymes was tested on S. aureus strain Newman and compared to both parental enzymes applied individually or simultaneously. After 10 rounds of sub-lethal exposure, parental LysK (two lytic domains) and lysostaphin (one lytic domain) yielded cultures with 42-fold and 585-fold increases in MICs, respectively. When S. aureus cells were simultaneously exposed to an equimolar concentration of LysK and lysostaphin, the final MIC increased 129-fold (FIG. 1b). In contrast, triple fusions K-L and L-K yielded cultures with a mere 8-fold and 2-fold increase, respectively (FIG. 1b, initial MICs are indicated in Table 1). Survivors of the ten round exposure to peptidoglycan hydrolase were passaged five times in medium with no peptidoglycan hydrolase added. These passaged bacteria retained their elevated MIC (data not shown), suggesting that the resistance was not physiologically-induced (e.g. selective conditioning), but was likely a result of genetic alterations. We compared our triple-acting fusions to small molecule antibiotics in similar assays. Trimethoprim/sulfamethoxazole and streptomycin were both assayed after 10 rounds of sub-lethal exposure and yielded 6-fold and 13-fold MIC increases, respectively (data not shown). Resistance development during 10 rounds of sub-lethal exposure in plate lysis assays showed an 8-fold increased resistance to lysostaphin, a 2-fold increase for LysK, and no detectable increase in resistance for triple fusions K-L or L-K (not shown).

### Example 6

### Intracellular S. aureus Eradication Assay/Peptidoglycan Hydrolases Harboring a Protein Transduction Domain Enhance Intracellular S. aureus Eradication

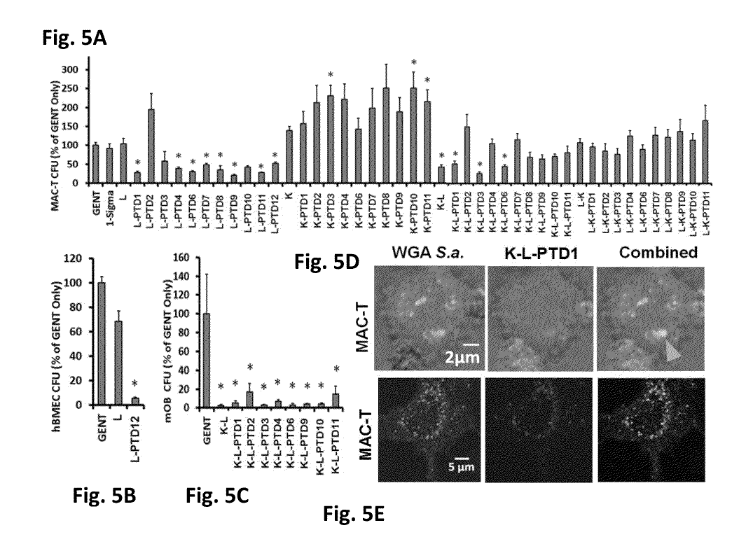

Protein Transduction Domains (PTDs) are cationic peptide sequences of ˜9-30 amino acids that occur naturally and facilitate protein transduction across eukaryotic cell membranes (Dietz, supra). Parental enzymes (lysostaphin, LysK) and triple fusions K-L and L-K were each modified by addition of 11 different C-terminal PTD sequences (Table 3; schematic FIG. 1a) and were tested for the ability to reduce intracellular S. aureus in multiple cultured cells known to support intracellular invasion (FIGS. 5a-5c).

Three similar intracellular S. aureus eradication assays from three different labs were optimized for (A) a cultured bovine mammary epithelial cell line (MAC-T, Nexia Biotechnologies, Quebec, Canada; Almeida, supra), (B) primary C57BL/6 mouse-derived osteoblasts (mOBs) (McCall et al. 2008. J. Bone Miner. Res. 23: 30-40) or (C) human brain microvascular endothelial cells (hBMEC; Stins, supra). MAC-T cells were grown in 24-well dishes, infected with 1.24 to 6.47×10⁷ S. aureus Newbould 305 (a known bovine mastitis causative agent) at an MOI of 28 to 209 for 2 hours at 37° C. in Dulbecco's Modified Eagle's Medium (DMEM) with no antibiotics. Osteoblasts were grown in 6 well plates as described earlier (Chauhan and Marriott. 2010. J. Med. Microbiol. 59:755-762) and infected with the S. aureus strain UAMS-1 at an MOI of 100:1 for 2 hours at 37° C. in osteoblast growth medium in the absence of antibiotics. HBMEC were cultured in the absence of antibiotics and infected with ˜1×10⁶ bacteria (M01=10) S. aureus strain ISP479C (Pattee, P. A. 1981. J. Bacteriol. 145:479-488) in RPMI culture media with 10% fetal bovine serum (FBS), for 2 h at 37° C. as described previously (Sheen, supra). Following 3×PBS washes to remove extracellular S. aureus, the infected eukaryotic cell cultures (A-C, above) were exposed to gentamicin (A) 100 μg/ml, 1 h (B) 50 μg/ml, 15 min, or (C) 100 μg/ml, 30 min to kill extracellular S. aureus. Subsequently, cells (A) and (C) were washed 3× with PBS to remove gentamicin, whereas cells (B) were cultured in the presence of 25 μg/ml gentamicin for 24 h. Purified peptidoglycan hydrolase proteins were added to (A) MAC-T cells at 25 μg in 1 ml DMEM for 2.5 h, to (B) mOBs at 5 μg/ml for 2 h, or to (C) the hBMECs at 5 μg/ml for 1 h. Gentamicin-only controls received (A) 100 μg/ml, (B) 25 μg/ml, or (C) 100 μg/ml gentamicin in place of the peptidoglycan hydrolase construct. The cultures were finally washed 3× with PBS and examined microscopically (to determine if there had been cell lysis), prior to being trypsinized with 0.05% trypsin+0.025% Triton X-100 in (A and C) PBS or (B) water. The cell lysate was subjected to serial dilution plating, and the resultant CFUs were counted and normalized to the gentamicin control for each assay. CFU numbers in the gentamicin only treated group were in the range of (A) 3.26×10⁴ to 6.19×10⁵, (B) 6-10×10³ or (C) 2-4×10⁵ per well. All analyses were performed with one factor ANOVA (α=0.05) and T-Test post hoc analysis with \check{S}idák correction to α, (α=0.05) for multiple comparisons, comparing each peptidoglycan hydrolase treatment to the gentamicin only treatment.

For Confocal Microscopy of MAC-T cells, a 10 ml TSB overnight culture of S. aureus strain Newbould was washed, resuspended in 10 ml DMEM without FBS, vortexed rigorously and pelleted prior to resuspension in fresh 10 ml DMEM without FBS to OD₆₀₀=0.2. 100 μl of 1 mg/ml AlexaFluor-488 conjugate of wheat germ agglutinin (WGA; Life Technologies) was added to 10 ml S. aureus 305 in DMEM. The cells were incubated at 37° C. for 30 min, then washed 6× and resuspended in DMEM without FBS.

Confocal fluorescent microscopy was performed for in situ detection of internalized S. aureus and peptidoglycan hydrolase particles in bovine MAC-T cells cultivated on uncoated 50 mm glass bottom dishes (MatTek Corporation, Ashland, Mass.) in high-glucose DMEM (HyClone, Logan, Utah) supplemented with 10% (v/v) heat-inactivated FBS (Atlanta Biologicals, Lawrenceville, Ga.) at 37° C. in a humidified 5% CO₂ incubator. After 18 h of culture, when MAC-T cells were about 90-95% confluent, the existing medium was removed and replaced with 1 ml DMEM without FBS containing AlexaFluor488-labeled S. aureus cells. Samples were incubated at 37° C. for 30 min, washed 6× with DMEM without FBS, and 1 ml media was added to each well of a 24-well plate. After 2 hours, MAC-T cells were washed 3× in DMEM without FBS, exposed to 200 μg/ml gentamicin in DMEM for 30 min and washed an additional 3×, to remove extracellular S. aureus. MAC-T cells were exposed to 50 μg/ml of construct K-L-PTD 1 labeled with AlexaFluor 610-X per the manufacturer's protocol (Life Technologies Corporation) for 2 h before 3×DMEM washes. DMEM was replaced with TL Hepes (bio.Lonza.com; solution #04-616) and a drop of NucBlue Fixed Cell Stain (Life Technologies Corporation) was applied to the MAC-T cells prior to imaging.

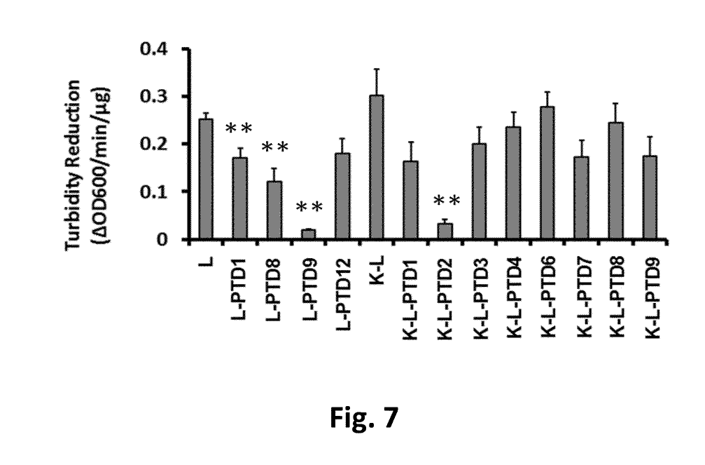

Neither commercial lysostaphin (1-Sigma; Sigma-Aldrich, St. Louis, Mo.) nor the C-terminal His₆-tagged lysostaphin (construct 1) were able to decrease the intracellular CFUs of S. aureus in a cultured bovine mammary epithelial cell line (MAC-T) or cultured human brain microvasculature endothelial cells (hBMEC) (FIGS. 5a, 5b). However, the addition of PTDs to the C-terminus of His₆-tagged lysostaphin resulted in significant reductions in intracellular S. aureus in both cell lines. In MAC-T cells, virtually all tested constructs displayed this effect, despite the fact that modification with PTDs often reduced the enzymatic activity of most constructs tested (FIG. 7). A striking example was the ability of construct 1-PTD12 to reduce the intracellular S. aureus strain ISP479C (Pattee, supra) from hBMEC cells in culture (FIG. 5b). In contrast, the ability of LysK or triple fusion L-K to eradicate intracellular S. aureus in MAC-T cells was inhibited by the addition of a PTD (FIG. 5a). Interestingly, triple fusion K-L reduced the intracellular bacteria recovered from either MAC-T cells or murine osteoblasts (mOB) and this effect was not significantly enhanced with the addition of a PTD (FIGS. 5a, 5c).

To confirm protein transfer across mammary epithelial cell membranes, cultured MAC-T cells were exposed to fluorescently-labeled K-L-PTD1 (red) and fluorescently-labeled S. aureus strain Newman (green) and monitored in real-time with confocal microscopy. S. aureus strain Newman and the triple fusion K-L-PTD1 were found to co-localize intracellularly in a single z-plane with MAC-T cells (FIG. 5d). A similar result was apparent in the maximum intensity projections (with all z-planes visualized) (FIG. 5e).

### Example 7

### Biofilm Eradication

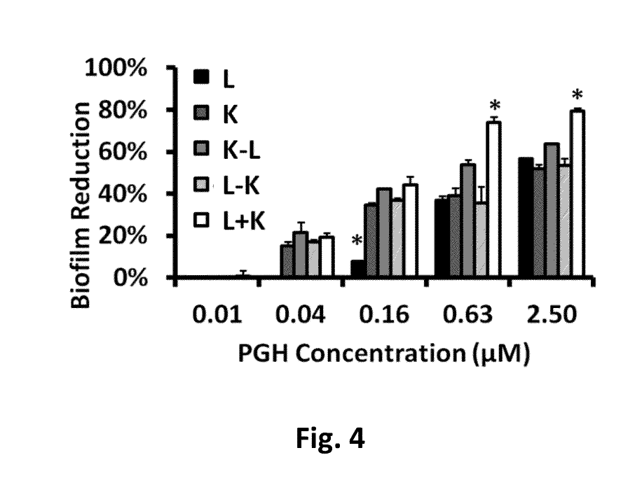

In the static biofilm reduction model, biofilms were produced as previously described (Gross et al. 2001. Infect. Immun. 69:3423-3426) with the following modifications. S. aureus strains were grown overnight in 5 ml TSB supplemented with 0.25% D(+)-glucose without shaking at 37° C. The culture was diluted 1/200 in fresh culture medium, and 200 μl was added to each well of a 96 well microtiter plate and incubated without shaking for 24 h at 37° C. Biofilms in the wells were washed twice with 200 μl SLB, treated with 50 μl peptidoglycan hydrolase (Lysostaphin, LysK and the triple fusion proteins K-L and L-K) in SLB at concentrations indicated (FIG. 4) for 1 h at room temperature, and washed twice with 100 μl SLB. Adherent bacteria were fixed with 200 μl 95% ethanol-5% glacial acetic acid for 20 min, washed once with water, and stained with 100 μl 0.4% crystal violet for 15 min at room temperature. Excess stain was removed with 3 water washes. Bound stain was resolubilized in 33% acetic acid, and 20 μl was transferred to a 96 well plate containing 180 μl water. The OD₅₉₀ₙₘ was determined in a Spectra Max plate reader. Percent reduction represents the difference in absorbance between biofilms exposed to buffer only and experimental wells that were exposed to the peptidoglycan hydrolases.

All proteins tested (L, K, K-L and L-K) effectively reduced staphylococcal biofilms when compared to buffer treated biofilms (FIG. 4). Static biofilms were created with S. aureus strain SA113 in 96 well dishes and treated for one hour with 100 μl of the indicated concentrations of each enzyme individually or as an equimolar mixture of LysK and lysostaphin. Each construct tested reduced the biofilm between 15 and 80% while the combination of LysK and lysostaphin at the higher concentrations tested, achieved slightly greater reductions that were significantly different compared to LysK alone.

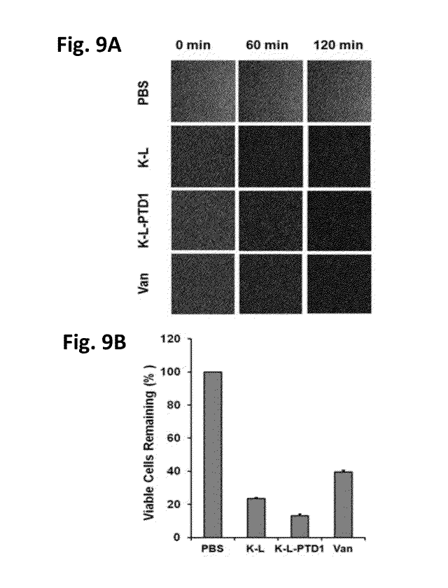

To determine whether modification with a PTD can impact biofilm clearance, triple fusion K-L, K-L-PTD1 and vancomycin were tested for antimicrobial activity in a dynamic biofilm model with MRSA strain NRS382 (FIG. 9). To develop dynamic S. aureus NRS382 biofilms, a 1:10 dilution of an overnight culture (TSB) was inoculated into a single-chamber or three-channel Stovall flow cell (IBI Scientific) with fresh TSB at 37° C. The inoculum was static for the first hour to allow initial bacterial attachment to the glass cover slip. The flow of fresh TSB was then started at a rate of 0.5 ml/min for up to 48 hours to allow for development of mature biofilms.

In order to visualize the viability of S. aureus NRS382 biofilms, each chamber of the flow cell was fluorescently labeled by the Live/Dead BacLight™ staining kit (Invitrogen, Carlsbad, Calif.) for one hour. The flow cells were then treated with either buffer (PBS) control, 100 μg/ml of vancomycin, triple fusion K-L or K-L-PTD1, all in PBS with a flow rate of 0.5 ml/min. At 0, 60, and 120 minutes, z-stacks of horizontal-plane images of biofilms were obtained with a 20×/1.3 objective lens by a Zeiss 710 confocal laser scanning microscope. The total z-stack image series contained 40×1 μm sections. To quantify the viability of S. aureus NRS382 biofilms at various time points in the presence of different treatments, one image at the center of z-series (i.e. center of the biofilm) for each time point was selected to represent each treatment group. Three squares representing 100×100 pixels were drawn in each image and the mean intensity of live (green channel) and dead (red channel) cell populations were calculated by Zen 2010 digital imaging software (Carl Zeiss). Percentage of live cells in the dynamic biofilm was calculated using a calibration “standard” curve that assigned a fluorescence intensity to live S. aureus NRS382 cells, over a range of cell concentrations. This allowed a direct translation of the mean fluorescent intensities to a percentage of live cells. The percentage of live cells remaining in biofilms after various treatments was statistically analyzed by an unpaired t-test.

At equal gram concentrations (100 μg/ml), all three had a pronounced effect on dynamic biofilms at 60 and 120 minutes post-treatment as visualized by Live/Dead™ viability staining (FIG. 9a). Despite a much reduced molar concentration compared to vancomysin [K-L (1.4 μM), K-L-PTD1 (1.4 μM), vancomycin (69 μM)], the peptidoglycan hydrolases were much more effective than vancomycin. Compared to controls (100% viable cells), vancomycin, triple fusion K-L, and K-L-PTD1 reduced the viability in dynamic biofilms to 40%, 24%, and 13%, respectively (FIG. 9b), with K-L-PTD1 being significantly more effective than K-L (FIG. 9b). This quantification was based on the 1 μm confocal microscopy sections depicted in FIG. 9a. Compiling mean fluorescent intensities from the entire biofilm z-stack (i.e., 40×1 μm slices) yielded similar results (not shown).

### Example 8

### Triple Fusion L-K Reduces Nasal Carriage of S. aureus

To determine whether our triple-acting fusion enzymes showed in vivo potency, we tested the parental and chimeric peptidoglycan hydrolases in a robust nasal colonization model. The rat nasal colonization model is an adaptation of the mouse nasal colonization model that we described previously (Schaffer et al. 2006. Infect. Immun. 74:2145-2153). All animal experiments were conducted in accordance with a protocol approved by the appropriate Institutional Animal Care and Use Committees. Wistar rats (5 to 6 wks of age) were obtained from Charles River Laboratories (Wilmington, Mass.) and given food and water ad libitum. The animals were housed two per cage in a modified barrier facility under viral antibody-free conditions. The rats were given drinking water containing streptomycin sulfate (0.5 g/liter; Sigma) 1 day prior to bacterial inoculation and for the course of the experiment. The drinking water and cages were changed three times per week. Rats were restrained briefly for inoculation in a DecapiCone bag (Braintree Scientific, Braintree, Mass.) and inoculated with 10 μl of 10⁷ CFU S. aureus strain ALR (Sm-resistant) on day 0. The rats were treated intranasally twice a day (6-7 h apart) on days 5, 6, and 7 with 200 μg of either lysostaphin (AMBI, Tarrytown, N.Y.) or recombinant peptidoglycan hydrolases in 20-μl buffer. Control rats received buffer alone. On day 10 the rats were euthanized; the area around the nasal region was wiped with 70% isopropyl alcohol, and the nasal tissue was excised and homogenized on ice in 600 μl TSB. Quantitative cultures of the homogenates were prepared by plating duplicate 100 μl aliquots on TSA plates containing 0.5 mg/ml Sm (to recover the S. aureus strain used for inoculation) and one blood agar plate (to evaluate the total nasal flora). The number of S. aureus colonies from quantitative plate counts was used to estimate the CFU/nose for each rat. The results from a three independent experiments were combined. For rat nasal colonization experiments, significant (P<0.05) differences between the median values of quantitative culture results for different rat groups were compared to the control group by the Mann-Whitney test (InStat; Graph Pad Software).

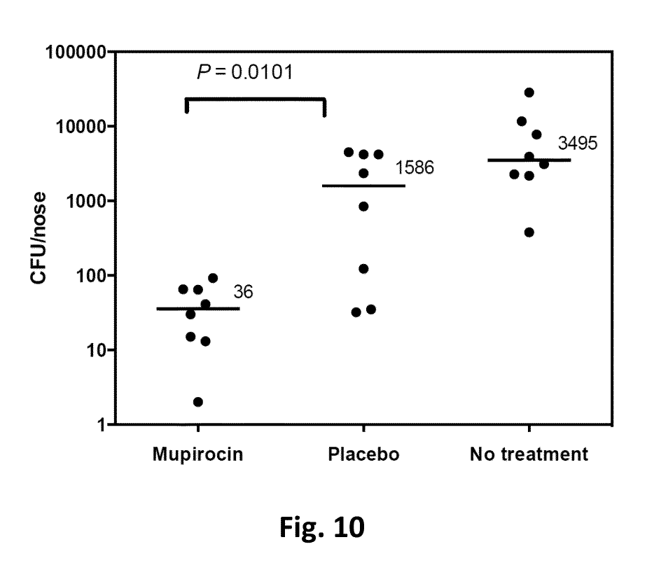

Treatment with triple fusion L-K resulted in a 98% decrease in the CFU/nose compared to rats treated with buffer (FIG. 1c). In contrast, equimolar concentrations of recombinant LysK, and triple fusion K-L were unable to significantly reduce the bacterial load. Rats treated with 200 μg lysostaphin (AMBI, Tarrytown, N.Y.) showed a 87% (p=0.085) reduction in nasal colonization consistent with previous findings (Walsh et al. 2004. Pharm. Res. 21:1770-1775). Colonies (N=22) recovered from the nares of rats treated with the triple fusion L-K were not more resistant than the parental strain in plate lysis analysis (not shown). The MIC of 8 post-treatment isolates toward triple fusion L-K was 7.0±2.4 μg/ml (N=32), similar to that of S. aureus ALR (9.4±6.1 μg/ml (N=8). A separate experiment in which rats were treated twice per day with 10 μl of 2% mupirocin resulted in a 98% reduction in nasal carriage (FIG. 10).

### Example 9

### Peptidoglycan Hydrolase-PTD Fusion Reduces S. aureus in a Murine Mastitis Model

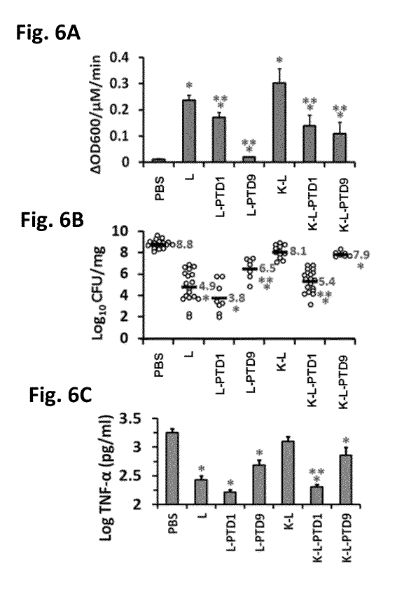

To determine if addition of a PTD domain impacts the ability of a peptidoglycan hydrolase to reduce the bacterial load in a murine model of mastitis, four constructs comprised of two different PTD domains fused to two different peptidoglycan hydrolase constructs (L-PTD1, 1-PTD9 and K-L-PTD1, K-L-PTD9) were chosen based on factors including purification yield, solubility, stability (not shown), and our results in MAC-T cells (FIG. 5). In turbidity reduction assays, triple fusion K-L was slightly more effective at lysing S. aureus strain Newbould 305 than lysostaphin, and the addition of PTD1 or PTD9 to either lysostaphin or triple fusion K-L reduced the in vitro activity (FIG. 6a).

To evaluate the efficacy of purified, individually administered peptidoglycan hydrolases in a murine model of bovine mastitis, female C57BL6/SJL mice were challenged by the intramammary route with 100 CFU S. aureus Newbould 305 in 50 μl buffer. A single 1.25 nmoles intramammary infusion of each desired peptidoglycan hydrolase (50 μl of a 25 μM solution) or 50 μl PBS was given 30 minutes post infection [with up to six glands treated per animal, each gland receiving a unique treatment] as described earlier (Schmelcher et al. 2012. Appl. Environ. Microbiol. 78:2297-2305; Kerr et al. 2001. Nat. Biotechnol. 19:66-70). Dams were euthanized 18 h post infusion, the mammary glands were aseptically dissected, and portions of the gland were used for determination of bacterial load and TNFα concentrations, as described previously (Schmelcher et al., supra).

In vivo, lysostaphin was effective in reducing the bacterial load approximately 4 logs compared to controls (FIG. 6b), and reduced the TNFα concentration in the mammary tissue >6-fold after challenge (FIG. 6c). Fusion of PTD1 to lysostaphin (L-PTD1) further decreased the mean bacterial load and TNFα concentration, but these results were not significantly different from lysostaphin lacking the PTD. Although the L-PTD9 fusion was virtually inactive in the turbidity reduction assay (FIG. 6a), it was still capable of reducing bacterial load within the mouse mammary glands relative to the PBS buffer control, although it was significantly less effective than either L-PTD1 or lysostaphin alone (FIGS. 6b, 6c). In contrast to our ex vivo data, triple fusion K-L was not able to clear the mammary gland bacterial infection more than buffer alone. However, S. aureus clearance was significantly enhanced when triple fusion K-L was fused to either PTD1 (K-L-PTD1) yielding clearance of the mammary gland to a level equivalent to that of lysostaphin alone or L-PTD1 (FIG. 6b, 6c).

Statistical analysis: The variables were analyzed as two-factor mixed models with Treatment as the factor and Mouse as random Blocks. This model ignores within mouse correlations. Log(TNFα) was used. The variance grouping technique was used for log CFU and log TNFα to correct for variance heterogeneity. Means comparisons were done with Sidak adjusted p-values so that the experiment-wise error was 0.05. Log(TNFα) is back-transformed to the original units for graphing.

### Example 10

### Triple Fusion K-L and K-L-PTD1 Reduce Intracellular S. aureus from Murine Calvaria

To examine the intracellular uptake and eradication in murine osteoblast-containing tissue samples, both ex vivo calvaria (skull cap), and in vivo femur injury models were tested. Calvaria were isolated from neonatal mice and infected with UAMS-1 strain of S. aureus (1×10⁶ bacteria/calvaria) for 2 h at 37° C. in osteoblast growth medium in the absence of antibiotics. Following 3× washes with PBS, calvaria were exposed to 50 μg/ml of gentamicin for 15 min to kill extracellular bacteria and further cultured in gentamicin containing osteoblast growth media. After 24 h later, calvaria were treated with peptidoglycan hydrolases (5 μg/ml) or gentamicin only control for 2 h. Lysis buffer containing 0.05% trypsin and 0.25% Triton X-100 was used to homogenize the calvaria, the homogenates were quantitatively plated on LB agar plates, and the CFU were enumerated after 24 h. The data represents triplicate determinants of two separate experiments.

Calvaria were isolated, and infected as described above using GFP-tagged UAMS-1 strain of S. aureus. Two hours post-infection, calvaria were washed with PBS followed by a 15 minutes treatment with 50 μg/ml gentamicin to kill extracellular bacteria. 24 hours post-infection, calvaria were treated with 5 μg/ml peptidoglycan hydrolases or gentamicin only control for two hours and embedded in Tissue-Tek O.C.T compound (Sakura, Torrance, Calif.). Tissues were sectioned and nuclei were counter stained with DAPI (Life technologies, Grand Island, N.Y.). GFP and DAPI levels of uninfected and infected sections were visualized under a fluorescent microscope at 20×. Fluorescence intensity from these sections was measured using ImageJ and defined as arbitrary fluorescence intensity units to quantify these results.

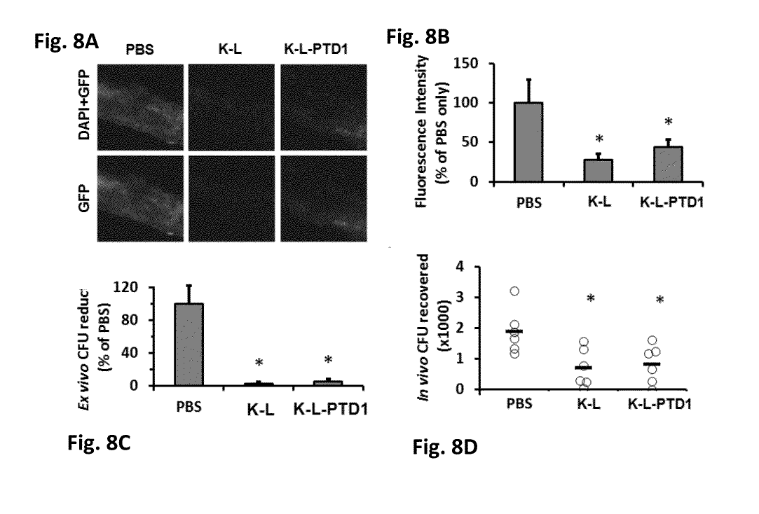

Both the triple fusion K-L and its PTD-modified version (K-L-PTD1) were able to eliminate GFP-labeled intracellular S. aureus strain UAMS-1 from murine calvaria (FIG. 8). Surviving S. aureus were detected by confocal microscopy of sectioned calvaria labeled with DAPI (FIG. 8a) and quantified (FIG. 8b). Both triple fusion K-L and K-L-PTD1 were able to reduce the number of live bacteria recovered from sectioned calvaria (FIG. 8c) and in a femur wound model (FIG. 8d) (Marriott et al., supra). These two animal models correlate well with the results in cultured osteoblasts in FIG. 5.

### Example 11

### Triple Fusion K-L and K-L-PTD1 Reduce Intracellular S. aureus Load in Murine Staphylococcal Osteomyelitis

We utilized a previously described (Marriott, supra) murine model of staphylococcal osteomyelitis that reproduces the clinical and gross pathological phases of inflammatory bone diseases, such as human post-traumatic osteomyelitis. C57BL/6J mice were anesthetized with isoflurane and the femur surgically exposed. A trough was drilled through the bone cortex by use of a high-speed drill with a round burr. Damaged bone sites were inoculated with S. aureus (1×10³) in agarose beads. Agarose beads containing S. aureus were prepared as follows: 1.4% low melt agarose (Invitrogen, Carlsbad, Calif.) was cooled to 40-42° C. prior to the addition of bacteria. This mixture was added to mineral oil, vigorously stirred, and cooled rapidly on ice. The resulting agarose beads were washed and stored on ice prior to bone application. This method of application induces local infection in bone tissue but markedly reduces the risk of systemic bacterial infection. The muscle fascia and surgical incision were closed, and the disease was allowed to proceed for 24 hours. Mice were either untreated or treated intramuscularly at the site of infection with peptidoglycan hydrolases (triple fusion K-L and K-L-PTD1) 5 mg/kg) given twice in a 24 hours period. Animals were euthanized, and the femurs were removed, homogenized in lysis buffer (0.05% trypsin and 0.25% Triton X-100 in water) and plated on LB agar plates. The data shown are the average normalized colony counts per ml of lysis buffer and represent the results of two separate experiments (n=6). Asterisks represent statistical significance as determined by one-way ANOVA followed by Tukey's posthoc test.

Both triple fusion K-L (p=0.012) and K-L-PTD1 (p=0.021) significantly reduced bacterial load as compared to no treatment, but the presence or absence of PTD1 had no significant effect (p=0.73) (FIG. 8d). Asterisks represent statistical significance as determined by one-way ANOVA followed by Tukey's posthoc test.

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

We claim:

1. A recombinant fusion nucleic acid encoding an antimicrobial Staphylococcus-specific triple fusion protein, wherein said encoded fusion protein comprises (1) a LysK endolysin CHAP endopeptidase domain, (2) a LysK endolysin amidase domain, and (3) a lysostaphin glycyl-glycine endopeptidase domain and a cell wall binding domain, wherein when in the K-L configuration, comprises: (1) a LysK endolysin CHAP endopeptidase domain, (2) a LysK endolysin amidase domain, (3) a lysostaphin glycyl-glycine endopeptidase domain, and cell wall binding domain, or when in the L-K configuration, comprises: (1) a lysostaphin glycyl-glycine endopeptidase domain, (2) a LysK endolysin-derived CHAP endopeptidase domain, (3) a LysK endolysin-derived amidase domain and a cell wall binding domain, said protein capable of cutting the peptidoglycan at three unique covalent bonds of the peptidoglycan simultaneously, with each domain being active independent of the others and each independent domain being capable of lysing the bacteria.

2. The recombinant fusion nucleic acid encoding the antimicrobial Staphylococcus-specific triple fusion protein of claim 1, wherein said encoded fusion protein has the sequence of SEQ ID NO:6 in the K-L configuration and SEQ ID NO:8 in the L-K configuration.

3. The recombinant fusion nucleic acid encoding the protein having the K-L configuration of claim 2, said nucleic acid having the sequence SEQ ID NO:5 and the nucleic acid encoding the protein having the L-K configuration of claim 2, said nucleic acid having the sequence SEQ ID NO:7.

4. The recombinant fusion nucleic acid of claim 1 further encoding a protein transduction domain (PTD), wherein addition of said PTD results in an encoded triple fusion protein K-L-PTD comprising (1) a LysK endolysin CHAP endopeptidase domain, (2) a LysK endolysin amidase domain, and (3) a lysostaphin glycyl-glycine endopeptidase domain and a cell wall binding domain and a protein transduction domain.

5. The recombinant fusion nucleic acid encoding the antimicrobial triple fusion protein of claim 4, wherein said encoded fusion protein has the sequence of SEQ ID NO:6 and further comprises the sequence of any one of sequences PTD-1 to 4 and 6-11 (SEQ ID NOs:54, 56, 58, 60, 62, 64, 66, 68, 70 and 72).

6. The fusion nucleic acid encoding K-L-PTD1, K-L-PTD2, K-L-PTD3, K-L-PTD4, K-L-PTD6, K-L-PTD7, K-L-PTD8, K-L-PTD9, K-L-PTD10 and K-L-PTD11, said fusion nucleic acid having the sequence SEQ ID NO:53, 55, 57, 59, 61, 63, 65, 67, 69, and 71, respectively.

7. A recombinant fusion nucleic acid encoding an antimicrobial lysostaphin fusion protein, comprising lysostaphin and a protein transduction domain (PTD), said fusion protein having lytic activity for the peptidoglycan cell wall resulting in bactericidal activity toward untreated, live intracellular and extracellular Staphylococcus aureus, including multi-drug resistant strains (e.g. MRSA).

8. The recombinant fusion nucleic acid encoding the lysostaphin-PTD protein of claim 7, wherein said encoded fusion protein is lysostaphin and any one of PTD1-PTD4 and PTD6-PTD12, said lysostaphin-PTD fusion proteins, L-PTD1, L-PTD2, L-PTD3, L-PTD4, L-PTD6, L-PTD7, L-PTD8, L-PTD9, L-PTD10, L-PTD11 and L-PTD12 having the sequence SEQ ID NOs:10, 12, 14, 16, 18, 20, 22, 24, 26, 28 and 30, respectively.

9. The fusion nucleic acid encoding L-PTD1, L-PTD2, L-PTD3, L-PTD4, L-PTD6, L-PTD7, L-PTD8, L-PTD9, L-PTD10, L-PTD11 and L-PTD12, said fusion nucleic acid having the sequence SEQ ID NO:9, 11, 13, 15, 17, 19, 21, 23, 25, 27 and 29, respectively.

10. A construct comprising the nucleic acid of any one of claims 1-9, wherein said nucleic acid is in operable linkage to a promoter that drives expression in a host cell.

11. A cloning vector comprising the construct of claim 10.

12. An expression vector comprising the construct of claim 10.

13. A process for transforming a host cell, comprising stably integrating the nucleic acid or the construct into the host cell.

14. A recombinant antimicrobial Staphylococcus-specific triple fusion protein, wherein said fusion protein comprises (1) a LysK endolysin CHAP endopeptidase domain, (2) a LysK endolysin amidase domain, and (3) a lysostaphin glycyl-glycine endopeptidase domain and a cell wall binding domain, wherein when in the K-L configuration, comprises: (1) a LysK endolysin CHAP endopeptidase domain, (2) a LysK endolysin amidase domain, (3) a lysostaphin glycyl-glycine endopeptidase domain, and cell wall binding domain, or when in the L-K configuration, comprises: (1) a lysostaphin glycyl-glycine endopeptidase domain, (2) a LysK endolysin-derived CHAP endopeptidase domain, (3) a LysK endolysin-derived amidase domain and a cell wall binding domain, said protein capable of cutting the peptidoglycan at three unique covalent bonds of the peptidoglycan simultaneously, with each domain being active independent of the others and each independent domain being capable of lysing the bacteria.

15. The fusion protein of claim 14, wherein said fusion protein has the sequence of SEQ ID NO:6 in the K-L configuration and SEQ ID NO:8 in the L-K configuration.

16. The recombinant fusion protein of claim 15 further comprising a protein transduction domain (PTD), wherein addition of said PTD results in a triple fusion protein comprising (1) a LysK endolysin CHAP endopeptidase domain, (2) a LysK endolysin amidase domain, and (3) a lysostaphin glycyl-glycine endopeptidase domain and a cell wall binding domain and a protein transduction domain.

17. The triple fusion protein of claim 16, wherein said encoded fusion protein has the sequence of SEQ ID NO:6 and further comprises the sequence of any one of sequences PTD-1 to 4 and 6-11.

18. The fusion proteins of claim 17, said fusion proteins K-L-PTD1, K-L-PTD2, K-L-PTD3, K-L-PTD4, K-L-PTD6, K-L-PTD7, K-L-PTD8, K-L-PTD9, K-L-PTD10 and K-L-PTD11, having the sequence of SEQ ID NOs: 54, 56, 58, 60, 62, 64, 66, 68, 70 and 72, respectively.

19. A lysostaphin-PTD fusion protein, wherein said fusion protein is lysostaphin and any one of PTD1-PTD4 and PTD6-PTD12, each lysostaphin-PTD fusion protein, L-PTD1, L-PTD2, L-PTD3, L-PTD4, L-PTD6, L-PTD7, L-PTD8, L-PTD, L-PTD10, L-PTD11 and L-PTD12 having the sequence SEQ ID NO:10, 12, 14, 16, 18, 20, 22, 24, 26, 28 and 30, respectively.

20. A LysK-PTD fusion protein, wherein said fusion protein is LysK and any one of PTD1-PTD4 and PTD6-PTD12, each LysK-PTD fusion protein, K-PTD1, K-PTD2, K-PTD3, K-PTD4, K-PTD6, K-PTD7, K-PTD8, K-PTD, K-PTD10, K-PTD11 and K-PTD12 having the sequence SEQ ID NO:32, 34, 36, 38, 40, 42, 44, 46, 48, 50 and 52, respectively.

21. A composition useful for the treatment of a disease caused by extracellular and intracellular S. aureus or multi-drug resistant strains (e.g. MRSA) wherein said composition comprises Lysostaphin (SEQ ID NO: 2) or a Lysostaphin-PTD fusion protein of claim 19 and a pharmaceutically acceptable carrier.

22. A composition useful for the treatment of a disease caused by extracellular and intracellular S. aureus or multi-drug resistant strains (e.g. MRSA) wherein said composition comprises LysK (SEQ ID NO:4) or a LysK-PTD fusion protein of claim 20 and a pharmaceutically acceptable carrier.

23. A composition useful for the treatment of a disease caused by extracellular and intracellular S. aureus or multi-drug resistant strains (e.g. MRSA) wherein said composition comprises the triple fusion protein of claim 14 or 15 in the K-L configuration or in the L-K configuration and a pharmaceutically acceptable carrier.

24. A composition useful for the treatment of a disease caused by extracellular and intracellular S. aureus or multi-drug resistant strains (e.g. MRSA) wherein said composition comprises any one of the triple fusion-PTD proteins (K-L-PTD) of claims 16-18 and a pharmaceutically acceptable carrier.

25. A method of treating an intracellular infection and disease caused by S. aureus or multi-drug resistant strains (e.g. MRSA) in an individual comprising:
administering to said individual an effective dosage of a composition of any one of claims 21, 23, and 24 wherein said composition comprises a recombinant antimicrobial Staphylococcus-specific lysostaphin-PTD fusion protein, the triple fusion K-L protein, or a K-L-PTD protein, said protein having lytic activity for the peptidoglycan cell wall resulting in bactericidal activity toward untreated, live intracellular and extracellular S. aureus, including multi-drug resistant strains (e.g. MRSA), wherein said administration is effective for the treatment of said staphylococci in multiple cell types.

26. A method of reducing nasal colonization by intracellular and extracellular S. aureus, including multi-drug resistant strains (e.g. MRSA) in an individual comprising:
administering to said individual an effective dosage of a composition of claim 23 comprising the triple fusion L-K protein or the composition of claim 21 comprising lysostaphin and a pharmaceutically acceptable carrier, wherein said administration is effective for the reduction of the nasal bacterial load in treated individuals when compared to untreated individuals.

27. The method of claim 26, wherein said triple fusion L-K protein has the sequence of SEQ ID NO:8.

28. A method of treating mastitis in an individual comprising:
administering intramammary treatments of an effective dosage of a composition comprising any one of claims 21 and 24 wherein said composition comprises a recombinant antimicrobial Staphylococcus-specific lysostaphin, lysostaphin-PTD fusion protein, or a K-L-PTD protein, wherein said administration is effective for reduction in the severity of said mastitis by reducing both the bacterial load and TNFα response by as much as 4 logs when compared to untreated individuals.

29. A method for removing or reducing a microbial biofilms, wherein said biofilm is a static biofilm caused by one or more of Staphylococcus, including multi-drug resistant strains (e.g. MRSA), comprising: contacting said biofilm with any effective dose of the composition of any one of claims 21, 22, 23 and 24 under conditions wherein the presence of said biofilm is reduced.

30. The method of claim 29, wherein the presence of said biofilm is significantly reduced after said contacting step.

31. The method of claim 29 wherein the microbial biofilms are present on a surface and are removed, eradicated or reduced from said surface.

32. A method of enhancing eradication of dynamic MRSA biofilms comprising:
treating S. aureus biofilms with an effective amount of the composition of any one of claims 23 and 24 comprising triple fusion K-L or K-L-PTD, wherein said treatment is effective in reducing the viability in dynamic biofilms as determined by viability measurements when compared to untreated biofilms.

33. A method of treating intracellular S. aureus osteoblast infections in calvaria of an individual comprising:
treating tissues with an effective amount of the compositions of claims 23 and 24 comprising triple fusion K-L and K-L-PTD1-12, and reducing the number of live intracellular S. aureus in treated calvaria tissue as compared to untreated tissue.

34. A method of treating staphylococcal osteomyelitis in an individual comprising:
administering intramuscularly at the site of infection an effective dosage of the compositions of claims 23 and 24 comprising triple fusion K-L and K-L-PTD1-12 for a time sufficient to cause significant osteomyelitis reduction within the individual, whereby the bacterial load is significantly reduced as compared to untreated individuals and systemic infection is absent.

35. A method of treating intracellular S. aureus infections in human brain microvascular endothelial cells (hBMEC) comprising treating hBMEC with an effective amount of a composition comprising lysostaphin-PTD12 fusion protein, whereby the number of live intracellular S. aureus is reduced in said cells as compared to untreated cells.
